(19) United States
(12) Patent Application Publication
(10) Pub. No.: US 2022/0265847 A1
(43) Pub. Date: Aug. 25, 2022

(54) METHODS AND COMPOSITIONS FOR TREATING NON-SMALL CELL LUNG CANCER

(71) Applicant: Board of Regents, The University of Texas System, Austin, TX (US)

(72) Inventors: John V. HEYMACH, Houston, TX (US); Monique NILSSON, Houston, TX (US)

(73) Assignee: Board of Regents, The University of Texas System, Austin, TX (US)

(21) Appl. No.: 17/611,019

(22) PCT Filed: May 14, 2020
(86) PCT No.: PCT/US2020/032968

§ 371 Date: Nov. 12, 2021

Related U.S. Application Data
(60) Provisional application No. 62/848,123, filed on May 15, 2019.

(51) Int. Cl. A61K 47/68; A61P 35/00; C07K 16/28; A61K 45/06; A61K 35/17

(52) U.S. Cl. CPC A61K 47/6857; A61P 35/00; C07K 16/2875; A61K 45/06; A61K 35/17; C07K 2317/24

(57) ABSTRACT

Aspects of the disclosure relate to a method for treating EGFR-mutant non-small-cell lung cancer (NSCLC) in a patient comprising administering a CD70 targeting molecule to the patient. Further aspects of the disclosure relate to a method for treating an epithelial-to-mesenchymal transition (EMT)-positive NSCLC in a patient comprising administering a CD70-targeting molecule to the patient.

### BACKGROUND OF THE INVENTION

This application claims priority of U.S. Provisional Application No. 62/848,123, filed on May 15, 2019, the entirety of which is incorporated herein by reference.

This invention was made with government support under grant number CA190628 awarded by the National Institutes of Health. The government has certain rights in the invention.

### 1. Field of the Invention

This invention relates to the field of molecular biology and medicine.

### 2. Background

Non-small-cell lung carcinoma (NSCLC) is any type of epithelial lung cancer other than small cell lung carcinoma (SCLC). NSCLC accounts for about 85% of all lung cancers. As a class, NSCLCs are relatively insensitive to chemotherapy, compared to small cell carcinoma. When possible, they are primarily treated by surgical resection with curative intent, although chemotherapy has been used increasingly both pre-operatively (neoadjuvant chemotherapy) and post-operatively (adjuvant chemotherapy).

EGFR mutant NSCLC patients are initially responsive to EGFR targeted therapies. However, resistant disease inevitably emerges, and in nearly half of resistance cases, tumors lack secondary EGFR mutations such as T790M and are refractory to 2nd and 3rd generation EGFR tyrosine kinase inhibitors (TKI). There is a need in the art for additional therapeutic approaches.

### SUMMARY OF THE INVENTION

Aspects of the disclosure relate to a method for treating EGFR-mutant non-small-cell lung cancer (NSCLC) in a patient comprising administering a CD70 targeting molecule to the patient. Further aspects of the disclosure relate to a method for treating an epithelial-to-mesenchymal transition (EMT)-positive NSCLC in a patient comprising administering a CD70-targeting molecule to the patient. Yet further aspects of the disclosure relate to a composition comprising a CD70 targeting molecule and one or more additional therapeutic agent(s).

In some embodiments, the patient has been determined to have EGFR-mutant NSCLC. In some embodiments, the NSCLC comprises lung adenocarcinoma. In some embodiments, the patient is a non-smoker. In some embodiments, the patient is a human.

The term EGFR mutant cancer refers to a cancer that has altered expression or activity of EGFR (epidermal growth factor receptor). The mutation may be in the coding region of EGFR and affect the expression levels of endogenous EGFR or the activity levels of the resulting protein. The mutation may also be in the non-coding portion of the gene, such as in the promoter region, 3′ or 5′ UTR, or intronic region. In some embodiments, the EGFR mutation is a gain of function mutation. In some embodiments, the EGFR mutation is a loss of function. In some embodiments, the EGFR mutation comprises an activating mutation. In some embodiments, the activating mutation comprises L858R. In some embodiments, the activating mutation comprises a deletion in exon 19. In some embodiments, the EGFR mutation comprises one or more of the following mutations instead of or in addition to these other mutations: G719S (c.2155G>A), G719C (c.2155G>T), G719A (c.2156G>C), S720F (c.2159C>T), Exon 19 deletion or partial deletion, D761Y (c.2281G>T), D770_N771 (insNPG), D770_N771 (insSVQ), D770_N771 (insG), V765A (c.2294T>C), T783A (c.2347A>G), S7681I (c.2303G>T), T790M (c.2369C>T), V769L (c.2305G>T), N771T (c.2312A>C), L858R (C.2573T>G), L861Q (c.2582T>A), L861R (c.2582T>G). In some embodiments, the EGFR mutation comprises a Class I, II, or III EGFR mutation. In some embodiments, the EGFR mutation comprises at least one of delE746-A750, delL747-P753insS, delL747-T751, delL747-A750insP, p.L747_S752del, K754insANKG, delT751_I759insN, delL747_A750insP, delE746_T751insV, delT751_I759insS, delE746_T751insI, delL747_A755insSKG, delE746_T751insVA, delL747_T751insP, delE746_S752insV, and delE746_A750insAP. In some embodiments, the EGFR mutation comprises at least one class I mutation selected from delE746-A750, delL747-P753insS, delL747-T751, delL747-A750insP, p.L747_S752del, K754insANKG, delT751_I759insN, delL747_A750insP, delE746_T751insV, delT751_I759insS, delE746_T751insI, delL747_A755insSKG, delE746_T751insVA, delL747_T751insP, delE746_S752insV, and delE746_A750insAP. In some embodiments, the EGFR mutation comprises an in-frame deletion or partial deletion in exon 19. In some embodiments, the EGFR mutation comprises at least one of G719S (c.2155G>A), G719C (c.2155G>T), G719A (c.2156G>C), S720F (c.2159C>T), D761Y (c.2281G>T), V765A (c.2294T>C), T783A (c.2347A>G), S7681I (c.2303G>T), T790M (c.2369C>T), V769L (c.2305G>T), N771T (c.2312A>C), L858R (C.2573T>G), L861Q (c.2582T>A), and L861R (c.2582T>G). In some embodiments, the EGFR mutation comprises at least one class II mutation selected from G719S (c.2155G>A), G719C (c.2155G>T), G719A (c.2156G>C), S720F (c.2159C>T), D761Y (c.2281G>T), V765A (c.2294T>C), T783A (c.2347A>G), S7681I (c.2303G>T), T790M (c.2369C>T), V769L (c.2305G>T), N771T (c.2312A>C), L858R (C.2573T>G), L861Q (c.2582T>A), and L861R (c.2582T>G). In some embodiments, the EGFR mutation comprises a single nucleotide substitution. In some embodiments, the EGFR mutation comprises at least one of D770_N771 (insNPG), D770_N771 (insSVQ), D770_N771 (insG). In some embodiments, the EGFR mutation comprises at least one class III mutation selected from D770_N771 (insNPG), D770_N771 (insSVQ), D770_N771 (insG). In some embodiments, the EGFR mutation comprises in-frame duplications or insertions in exon 20. It is contemplated that at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 (or any range derivable therein) of these mutations may be determined, known, or used in embodiments described herein. In specific embodiments, 1, 2, 3, 4, 5, 6, 7, 8, 9, 10 or more of these mutations may be excluded in an embodiment.

In some embodiments, the patient has not been tested for CD70 expression in cancer cells from the patient. In some embodiments, the patient has been determined to have CD70-expressing cancer cells. In some embodiments, the patient has been previously treated for NSCLC. In some embodiments, the patient has been determined to have acquired resistance to the previous treatment. In some embodiments, the previous treatment comprises EGFR tyrosine kinase inhibitor (TKI) therapy and wherein the therapy comprises one or more EGFR TKIs. In some embodiments, the previous treatment comprises single-agent EGFR TKI therapy. In some embodiments, the previous treatment comprises a combination of at least two EGFR TKIs. In some embodiments, the patient has been determined to be resistant to at least two EGFR TKIs. In some embodiments, was determined to have systemic disease progression while receiving continuous EGFR TKI therapy. In some embodiments, the EGFR TKI therapy comprises one or more of gefitinib, erlotinib, afatinib, dacomitinib, osimertinib, and brigatinib. In some embodiments, the EGFR TKI therapy comprises one or more of erlotinib, gefitinib, and osimertinib. In some embodiments, the EGFR TKI therapy comprises at least 2 of gefitinib, erlotinib, afatinib, dacomitinib, osimertinib, and brigatinib. In some embodiments, the EGFR TKI therapy comprises at least three of gefitinib, erlotinib, afatinib, dacomitinib, osimertinib, and brigatinib. In some embodiments, the EGFR TKI therapy comprises at least four of gefitinib, erlotinib, afatinib, dacomitinib, osimertinib, and brigatinib. It is specifically contemplated that one or more of these may be excluded as the EGFR TKI therapy.

In some embodiments, the method further comprises administration of an additional therapy or the composition comprises an additional therapeutic agent. In some embodiments, the additional therapy or agent comprises chemotherapy, radiation, surgery, TKI therapy, or an immunotherapy. In some embodiments, the additional therapy or agent comprises one or more of durvalumab, atezolizumab, pembrolizumab, nivolumab, necitumumab, and bevacizumab. In some embodiments, the additional therapy or agent comprises one or more of carboplatin, pemetrexed, nab-paclitaxel, photofrin, cisplatin, docetaxel, gemcitabine, paclitaxel, and vinorelbine. In some embodiments, the additional therapy or agent comprises one or more of alectinib, lorlatinib, and ceritinib. In some embodiments, the additional therapy or agent comprises one or more of gefitinib, erlotinib, afatinib, dacomitinib, osimertinib, brigatinib, and combinations thereof. In some embodiments, the additional therapy or agent comprises osimertinib. In some embodiments, the method further comprises administration of adjuvant and/or neo-adjuvant therapy. In some embodiments, the additional therapy may be conjugated or linked to the CD70 targeting therapy. In some embodiments, the linkage is through a chemical linker. In some embodiments, the linkage is through a peptide bond (eg. a fusion protein comprising a CD70 targeting agent an an additional therapeutic agent).

In some embodiments, the patient has been determined to be ALK mutant. In some embodiments, the patient has been determined to not be ALK mutant.

In some embodiments, the CD70 targeting molecule comprises an anti-CD70 antibody or a CD70-binding fragment thereof. In some embodiments, the antibody is humanized or chimeric. In some embodiments, the antibody is conjugated to a molecule. In some embodiments, the antibody is conjugated to a toxic molecule. In some embodiments, the toxic molecule comprises monomethyl auristatin E (MMAE), monomethyl auristatin F (MMAF), Pyrrolobenzodiazepine (PBD), duocarmycin, or combinations thereof.

In some embodiments, the CD70 targeting molecule comprises a heavy chain variable region and/or a light chain variable region from a CD70 antibody. In some embodiments, the CD70 targeting molecule comprises a CDR1, CDR2, and CDR3 from a heavy chain variable region and/or a CDR1, CDR2, and CDR3 from a light chain variable region. The heavy chain and light chain variable regions may be from or derived from a CD70 antibody. In some embodiments, the CD70 targeting molecule comprises a single chain variable fragment (scFV). The scFv may comprise hypervariable regions, such as CDR1, CDR2, and CDR3 from a heavy chain variable region and/or a CDR1, CDR2, and CDR3 from a light chain variable region from an anti-CD70 antibody. In some embodiments, the antibody comprises cusatuzumab or vorsetuzumab. In some embodiments, the CD70 targeting molecule comprises a heavy chain variable region and/or a light chain variable region from cusatuzumab or vorsetuzumab. In some embodiments, the CD70 targeting molecule comprises a CDR1, CDR2, and CDR3 from the heavy chain variable region of cusatuzumab and/or a CDR1, CDR2, and CDR3 from the light chain variable region of cusatuzumab. In some embodiments, the CD70 targeting molecule comprises a CDR1, CDR2, and CDR3 from the heavy chain variable region of vorsetuzumab and/or a CDR1, CDR2, and CDR3 from the light chain variable region of vorsetuzumab.

In some embodiments, the additional therapy or agent in the methods and compositions of the disclosure comprises a secondary antibody linked to a toxic molecule. For example, the combination therapy may comprise a CD70 targeting antibody in combination with an agent comprising a conjugate comprising a secondary antibody that binds to the CD70 targeting antibody conjugated to a toxic molecule. Accordingly, such combination of agents delivers an antibody linked through a secondary antibody to a toxic molecule. In some embodiments, the secondary antibody and toxic molecule are linked through a cleavable linker.

In some embodiments, the CD70 targeting molecule comprises a bi-specific T cell engager (BiTE), a chimeric antigen receptor (CAR), a T cell comprising a CAR, or a tri-specific natural killer cell engager therapy (TriNKET). In some embodiments, the BiTE, CAR, or TriNKET is derived from the heavy chain variable regions and/or light chain variable regions of cusatuzumab or vorsetuzumab. In some embodiments, the BiTE, CAR, or TriNKET comprises a heavy chain comprising CDR1, CDR2, and CDR3 of cusatuzumab or vorsetuzumab and/or a light chain comprising CDR1, CDR2, and CDR3 of cusatuzumab or vorsetuzumab. In some embodiments, the CD70 targeting molecule comprises cusatuzumab-MMAE, vorsetuzumab-MMAE, or combinations thereof. In some embodiments, the CD70 targeting molecule comprises SGN-75, SGN-CD70A, AMG 172, and/or ARGX-110. SGN-75 is a CD70-blocking IgG1 antibody-drug conjugate (ADC) that releases its cell-killing agent upon internalization into CD70-expressing tumor cells. SGN-CD70A comprises a CD70-blocking antibody, equipped with a cytotoxic agent. SGN-CD70A comprises a highly potent cytotoxic, a pyrrolobenzodiazepine dimer, stably linked to a CD70-directed antibody. AMG 172 is an IgG1 ADC of which binding and internalization into CD70-expressing tumor cells induces metaphase arrest, followed by cellular apoptosis and eventually tumor cell death. ARGX-110 comprises a CD70-blocking IgG1 monoclonal antibody (mAb) whose glyoengineered Fc domain mediates targeted killing of CD70-expressing tumor cells via complement-dependent cytotoxicity (CDC), antibody-dependent cellular phagocytosis (ADCP) properties and enhanced antibody-dependent cellular cytotoxicity (ADCC). In some embodiments, the a biological sample from the patient has been determined to be positive for one or more EMT markers. In some embodiments, the one or more EMT markers comprise a reduction of an epithelial marker and/or an increase of a mesenchymal marker. In some embodiments, the EMT markers comprise 1, 2, 3, 4,or all 5 of CDH1, VIM, AXL, ZEB1, and ZEB2. In some embodiments, the biological sample comprises tumor cells and/or tumor-associated cells.

CD70 targeting molecules useful in the methods and compositions of the disclosure are known in the art. For example, CD70 CARs have been developed and can be used in embodiments of the disclosure. Accordingly, In some embodiments, the CD70 targeting molecule comprises CTX130. CTX130 is an allogeneic CRISPR/Cas9 gene-edited CAR-T cell therapy targeting CD70 made by CRISPR Therapeutics. In some embodiments, the CD70 targeting molecule comprises ALLO-316. ALLO-316 is an anti-CD70 AlloCAR T cell therapy that is being developed by Allogene. Further embodiments are described in Wang, Q J. et al., Clin Cancer Res. 2017 May 1; 23(9):2267-2276, which is herein incorporated by reference. It is contemplated that the CD70 targeting molecules described as useful for other indications may be used in the method and composition embodiments of the current disclosure. In some embodiments, the CD70 targeting molecule comprises a CD70 ligand, such as CD27. In some embodiments, the CD70 targeting molecule comprises a truncated CD27. In some embodiments, the CD70 targeting molecule comprises a CD27 CAR, which is a CD27 polypeptide fused to the transmembrane region and intracellular signaling region of a CAR molecule, such as 41BB and CD3-zeta. The CD27 polypeptide may be full length polypeptide or a fragment or truncated version thereof that interacts and binds to CD70. The CARs of the disclosure may be expressed on T cells or NK cells.

In some embodiments, the CD70 targeting molecule comprises a cell comprising a BiTE, CAR, or TriNKET. In some embodiments, the cell comprises a stem cell, a progenitor cell, an immune cell, or a natural killer (NK) cell. In some embodiments, the cell comprises a hematopoietic stem or progenitor cell, a T cell, a cell differentiated from mesenchymal stem cells (MSCs) or an induced pluripotent stem cell (iPSC). In some embodiments, the cell is isolated or derived from peripheral blood mononuclear cell (PBMCs). In some embodiments, the T cell comprises a cytotoxic T lymphocyte (CTL), a CD8⁺ T cell, a CD4⁺ T cell, an invariant NK T (iNKT) cell, a gamma-delta T cell, a NKT cell, or a regulatory T cell.

In some embodiments, the biological sample comprises a biopsy. In some embodiments, the biological sample is one obtained by methods such fine needle aspiration, core needle biopsy, vacuum assisted biopsy, incisional biopsy, excisional biopsy, punch biopsy, shave biopsy or skin biopsy. In certain embodiments the sample is obtained from a biopsy from lung tissue by any of the biopsy methods previously mentioned. In other embodiments the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional. A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen. The sample may be obtained by methods known in the art. In certain embodiments the samples are obtained by biopsy.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell biology to indicate that a value includes the standard deviation of error for the device or method being employed to determine the value.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. HCC827 and HCC4006 (both EGFR mutant) cells are sensitive to EGFR TKIs including erlotinib and osimertinib compared to EGFR TKI resistant (ER) variants.

FIG. 2A-H. EGFR TKI resistance is associated with transcriptomic alterations, epithelial to mesenchymal transition (EMT) and upregulation of CD70. (A) The inventors compared gene expression by RNA seq between parental HCC827 and HCC4006 cells and cells with acquired resistance to EGFR TKIs. (B) Gene Set enrichment analysis reveals an increased EMT signature in EGFR TKI resistant (ER) cells compared to parental cells. ER cells expressed lower levels of the epithelial marker CDH1 (C) and increased expression of the mesenchymal markers VIM, AXL, ZEB1 and ZEB2 (D-G). (H) EGFR TKI resistant cells significantly upregulated expression of CD70.

FIG. 3. Protein levels of CD70 are elevated on the surface of EGFR TKI resistant NSCLC cells. Flow cytometry reveals increased protein levels of CD70 on EGFR TKI resistant cells HCC827 ER1, ER3 and ER6 compared to HCC827 parental cells.

FIG. 4A-D. Induction of EMT is sufficient to induce EGFR TKI resistance. (A) Induced expression of ZEB1 in HCC827 (EGFR TKI sensitive) cells resulted in a shift to a mesenchymal phenotype as demonstrated by loss of E-cadherin and increased expression of N-cadherin, Axl, and vimentin. ZEB1 expression rendered HCC827 cells resistant to EGFR TKIs erlotinib, osimertinib, and afatinib (B-D).

FIG. 5A-B. In human NSCLC, EMT is associated with high expression of CD70. CD70 expression is significantly associated with an EMT gene expression signature in NSCLC cell lines (A) and clinical specimens from TCGA (B).

FIG. 6A-D. EGFR TKI resistance is associated with epithelial to mesenchymal transition (EMT). (A) The inventors compared gene expression by RNA seq between parental HCC827 and HCC4006 cells and cells with acquired resistance to EGFR TKIs. Gene set enrichment analysis reveals an EMT signature in EGFR TKI resistant (ER) cells compared to parental cells. ER cells expressed lower levels of the epithelial marker CDH1 and increased expression of the mesenchymal markers VIM, AXL, ZEB1 and ZEB2 (B). (C&D) EGFR TKI resistant cells including erlotinib resistant (ER) and osimertinib resistant (OR) cells displayed a proteomic signature consistent with EMT as determined by reverse phase protein array.

FIG. 7A-F. CD70 is elevated in T790M negative EGFR TKI resistant cells. (A) RNAseq analysis revealed overexpression of CD70 in EGFR TKI resistant cells compared to parental EGFR TKI sensitive cells. (B-F) Cell surface expression of CD70 was evaluated by flow cytometry. CD70 expression was elevated in EGFR TKI resistant cells including erlotinib resistant (ER) and osimertinib resistant (OR) lines compared to the parental EGFR TKI-sensitive cell lines (HCC4006, HCC827, H1975). CD70 expression was minimal in cells where acquired resistance to EGFR TKIs was mediated by secondary EGFR mutations (T790M) or MET amplification.

FIG. 8. CD70 is elevated in genetically engineered mouse models (GEMM) of EGFR TKI resistance. To generate mouse models of acquired EGFR independent NSCLC, doxycycline (DOX) was used to induce EGFR mutant NSCLC tumors in a DOX-inducible L858R EGFR mouse model. Once tumors were visible by CT imaging, DOX was withdrawn from a subset of animals. After a period of tumor regression, resistant tumors began to re-grow. Animals were euthanized and tumors collected for immunohistochemical staining for CD70. CD70 expression was elevated in tumors that acquired EGFR independence.

FIG. 9. CD70 is elevated in NSCLC clinical specimens following EGFR TKI resistance. Immunohistochemical staining of CD70 revealed minimal CD70 expression on EGFR TKI treatment naïve EGFR mutant NSCLC tumors. However, CD70 expression was markedly elevated on EGFR mutant tumors after therapeutic resistance to EGFR TKI treatment.

FIG. 10A-C. Induction of EMT is sufficient to induce EGFR TKI resistance. (A) Induced expression of ZEB1 in HCC827 (EGFR TKI sensitive) cells resulted in a shift to a mesenchymal phenotype as demonstrated by loss of E-cadherin and increased expression of N-cadherin, Axl, and vimentin. (B) ZEB1 expression rendered HCC827 cells resistant to EGFR TKIs erlotinib, osimertinib, and afatinib. (C) ZEB1 expression induce a significant rise in CD70 mRNA expression and cell surface expression of CD70.

FIG. 11A-B. In human NSCLC clinical specimens and NSCLC cell lines, EMT is associated with high expression of CD70. CD70 expression is significantly associated with an EMT gene expression signature and ZEB1 in NSCLC clinical specimens from TCGA (A) and NSCLC cell lines (B).

FIG. 12. Stimulation of CD70 activates signal transduction pathways in EGFR TKI resistant cells. HCC4006 osimertinib-resistant (OR) cells were treated with soluble CD27, the binding partner of CD70, and the effect on downstream signal transduction pathways was evaluated by Western blotting. CD70 activation resulted in phosphorylation of Akt and ERK.

FIG. 13A-C. Knockdown of CD70 impairs the growth of EGFR TKI resistant cells. siRNA mediated knockdown of CD70 (A) resulted in impaired viability of H1975 OR5 cells (B). Similar results were obtained with HCC4006 OR cells (C).

FIG. 14A-C. CD70 antibody-drug conjugates (ADCs) target CD70+ EGFR TKI resistant cell in vitro. CD70 ADCs cuzatuzumab-MMAE and vorsetuzumab-MMAE demonstrated anti-tumor cell activity against H1975 osimertinib resistant (OR) cells including H1975 OR5 and H1975 OR16. (A) shows the percent CD70 positive cells in various cell lines, and the line graphs show the relative cell viability in the cell lines in with the addition of (B) cuzatuzumab-MMAE and (C) vorsetuzumab-MMAE.

FIG. 15. CD70 ADCs enhance the activity of osimertinib (OSI) in EGFR TKI resistant cells in vitro. The bar graphs show the relative cell viability of the combination of OSI and the the ADCs.

FIG. 16. CD70 ADCs have activity against EGFR TKI resistant cells in vitro. CD70 ADCs cuzatuzumab-MMAE and vorsetuzumab-MMAE demonstrated anti-tumor cell activity against H1975 osimertinib resistant (OR) cells including.

### DESCRIPTION OF ILLUSTRATIVE EMBODIMENTS

While EGFR mutant NSCLC patients are initially responsive to EGFR tyrosine kinase inhibitors (TKI), resistant disease inevitably emerges. The current disclosure provides novel therapeutic approaches to NSCLC. Further embodiments are described below.

### I. DEFINITIONS

The terms “protein,” “polypeptide,” and “peptide” are used interchangeably herein when referring to a gene product.

“Homology,” or “identity” refers to sequence similarity between two peptides or between two nucleic acid molecules. Identity can be determined by comparing a position in each sequence which may be aligned for purposes of comparison. When a position in the compared sequence is occupied by the same base or amino acid, then the molecules share sequence identity at that position. A degree of identity between sequences is a function of the number of matching or homologous positions shared by the sequences. An “unrelated” or “non-homologous” sequence shares less than 60% identity, less than 50% identity, less than 40% identity, less than 30% identity, or less than 25% identity, with one of the sequences of the current disclosure.

The terms “amino portion,” “N-terminus,” “amino terminus,” and the like as used herein are used to refer to order of the regions of the polypeptide. Furthermore, when something is N-terminal to a region it is not necessarily at the terminus (or end) of the entire polypeptide, but just at the N-terminus of the region or domain. Similarly, the terms “carboxy portion,” “C-terminus,” “carboxy terminus,” and the like as used herein is used to refer to order of the regions of the polypeptide, and when something is C-terminal to a region it is not necessarily at the terminus (or end) of the entire polypeptide, but just at the C-terminus of the region or domain.

The terms “polynucleotide,” “nucleic acid,” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, dsRNA, siRNA, miRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

Cells or a culture of cells are “substantially free” of certain reagents or elements, such as serum, signaling inhibitors, animal components or feeder cells, exogenous genetic elements or vector elements, as used herein, when they have less than 10% of the element(s), and are “essentially free” of certain reagents or elements when they have less than 1% of the element(s). However, even more desirable are cell populations wherein less than 0.5% or less than 0.1% of the total cell population comprise exogenous genetic elements or vector elements.

Cells or a culture of cells are “essentially free” of certain reagents or elements, such as serum, signaling inhibitors, animal components or feeder cells, when the culture, matrix or medium respectively have a level of these reagents lower than a detectable level using conventional detection methods known to a person of ordinary skill in the art or these agents have not been extrinsically added to the culture, matrix or medium. The serum-free medium may be essentially free of serum.

A “gene,” “polynucleotide,” “coding region,” “sequence,” “segment,” “fragment,” or “transgene” which “encodes” a particular protein, is a nucleic acid molecule which is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “cell” is herein used in its broadest sense in the art and refers to a living body which is a structural unit of tissue of a multicellular organism, is surrounded by a membrane structure which isolates it from the outside, has the capability of self-replicating, and has genetic information and a mechanism for expressing it. Cells used herein may be naturally-occurring cells or artificially modified cells (e.g., fusion cells, genetically modified cells, etc.).

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

In some embodiments, the methods are useful for reducing the size and/or cell number of a tumor. In some embodiments, the method of the disclosure are useful for inhibiting the growth of tumors, such as solid tumors, in a subject.

The term “antibody” includes monoclonal antibodies, polyclonal antibodies, dimers, multimers, multispecific antibodies and antibody fragments that may be human, mouse, humanized, chimeric, or derived from another species. A “monoclonal antibody” is an antibody obtained from a population of substantially homogeneous antibodies that is being directed against a specific antigenic site.

“Antibody or functional fragment thereof means an immunoglobulin molecule that specifically binds to, or is immunologically reactive with a particular antigen or epitope, and includes both polyclonal and monoclonal antibodies. The term antibody includes genetically engineered or otherwise modified forms of immunoglobulins, such as intrabodies, peptibodies, chimeric antibodies, fully human antibodies, humanized antibodies, and heteroconjugate antibodies (e.g., bispecific antibodies, diabodies, triabodies, and tetrabodies). The antibody may be derived from natural sources, or partly or wholly synthetically produced. An antibody may be monoclonal or polyclonal. The antibody may be a member of any immunoglobulin class, including any of the human classes: IgG, IgM, IgA, IgD, and IgE. The term functional antibody fragment includes antigen binding fragments of antibodies, including e.g., Fab′, F(ab′)₂, Fab, Fv, rlgG, and scFv fragments. The term scFv refers to a single chain Fv antibody in which the variable domains of the heavy chain and of the light chain of a traditional two chain antibody have been joined to form one chain. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment retains the ability to bind its cognate antigen at comparable affinity to the full antibody.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this disclosure. In contrast to polyclonal antibody preparations, which typically include several different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The phrases “pharmaceutical composition” or “pharmacologically acceptable composition” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, and Ringer's dextrose), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition may be adjusted according to well-known parameters.

The term “unit dose” or “dosage” refers to physically discrete units suitable for use in a subject, each unit containing a predetermined quantity of the therapeutic composition calculated to produce the desired responses discussed herein in association with its administration, i.e., the appropriate route and treatment regimen. The quantity to be administered, both according to number of treatments and unit dose, depends on the effect desired. The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 μg/kg/body weight to about 1000 mg/kg/body weight (this such range includes intervening doses) or more per administration, and any particular dose derivable therein. In non-limiting examples of a range derivable from the numbers listed herein, a range of about 5 μg/kg/body weight to about 100 mg/kg/body weight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject.

The use of a single chain variable fragment (scFv) is of particular interest. scFvs are recombinant molecules in which the variable regions of light and heavy immunoglobulin chains encoding antigen-binding domains are engineered into a single polypeptide. Generally, the V_{H} and V_{L} sequences are joined by a linker sequence. See, for example, Ahmad (2012) Clinical and Developmental Immunology Article ID 980250, herein specifically incorporated by reference. Described herein are BCMA-specific scFv molecules that comprise the variable regions of light and heavy immunoglobulin chains encoding BCMA-binding domains that are engineered into a single polypeptide. Similarly, the CS1-specific scFv molecules described herein comprise the variable regions of light and heavy immunoglobulin chains encoding CS1-binding domains that are engineered into a single polypeptide.

As used herein, the term “binding affinity” refers to the equilibrium constant for the reversible binding of two agents and is expressed as a dissociation constant (Kd). Binding affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more (or any derivable range therein), than the binding affinity of an antibody for unrelated amino acid sequences. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges.

A “therapeutically effective amount” or “efficacious amount” refers to the amount of an agent, or combined amounts of two agents, that, when administered to a mammal or other subject for treating a disease, is sufficient to effect such treatment for the disease. The “therapeutically effective amount” will vary depending on the agent(s), the disease and its severity and the age, weight, etc., of the subject to be treated.

Subject” and “patient” refer to either a human or non-human, such as primates, mammals, and vertebrates. In particular embodiments, the subject is a human.

Throughout this application, the term “about” is used to indicate that a value includes the inherent variation of error for the device, the method being employed to determine the value, or the variation that exists among the study subjects.

### II. CD70 TARGETING AGENTS

A. Antibodies

Aspects of the disclosure relate to CD70 targeting agents. In some embodiments, the CD70 targeting agent comprises an anti-CD70 antibody or a fragment thereof. The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986 See, e.g., Epitope Mapping Protocols, supra. Additionally, antigenic regions of proteins can also be predicted and identified using standard antigenicity and hydropathy plots.

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al. Front Immunol. 2013; 4: 302; 2013)

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (κ) and lambda (λ). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the —COOH end. The isotype of an antibody can be IgM, IgD, IgG, IgA, or IgE and is defined by the heavy chains present of which there are five classifications: mu (μ), delta (δ), gamma (γ), alpha (α), or epsilon (ε) chains, respectively. IgG has several subtypes, including, but not limited to, IgG1, IgG2, IgG3, and IgG4. IgM subtypes include IgM1 and IgM2. IgA subtypes include IgA1 and IgA2.

Antibodies can be whole immunoglobulins of any isotype or classification, chimeric antibodies, or hybrid antibodies with specificity to two or more antigens. They may also be fragments (e.g., F(ab′)2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins, such as the following:

The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bi-specific, meaning the two antigen-binding sites have different antigen specificities.

Bispecific antibodies are a class of antibodies that have two paratopes with different binding sites for two or more distinct epitopes. In some embodiments, bispecific antibodies can be biparatopic, wherein a bispecific antibody may specifically recognize a different epitope from the same antigen. In some embodiments, bispecific antibodies can be constructed from a pair of different single domain antibodies termed “nanobodies”. Single domain antibodies are sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as scFv. Diabodies and scFvs can be constructed without an Fc region, using only variable domains, potentially reducing the effects of anti-idiotypic reaction. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

In certain aspects, the antigen-binding domain may be multispecific or heterospecific by multimerizing with VH and VL region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

In some embodiments, multispecific antibodies can be used and directly linked via a short flexible polypeptide chain, using routine methods known in the art. One such example is diabodies that are bivalent, bispecific antibodies in which the VH and VL domains are expressed on a single polypeptide chain, and utilize a linker that is too short to allow for pairing between domains on the same chain, thereby forcing the domains to pair with complementary domains of another chain creating two antigen binding sites. The linker functionality is applicable for embodiments of triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001)).

Bispecific diabodies, as opposed to bispecific whole antibodies, may also be advantageous because they can be readily constructed and expressed in E. coli. Diabodies (and other polypeptides such as antibody fragments) of appropriate binding specificities can be readily selected using phage display (WO94/13804) from libraries. If one arm of the diabody is kept constant, for instance, with a specificity directed against a protein, then a library can be made where the other arm is varied and an antibody of appropriate specificity selected. Bispecific whole antibodies may be made by alternative engineering methods as described in Ridgeway et al., (Protein Eng., 9:616-621, 1996) and Krah et al., (N Biotechnol. 39:167-173, 2017), each of which is hereby incorporated by reference in their entirety.

Heteroconjugate antibodies are composed of two covalently linked monoclonal antibodies with different specificities. See, e.g., U.S. Pat. No. 6,010,902, incorporated herein by reference in its entirety.

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, VH and VL, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the VL domain are identified as L1, L2, and L3, with L1 occurring at the most distal end and L3 occurring closest to the CL domain. The CDRs may also be given the names CDR-1, CDR-2, and CDR-3. The L3 (CDR-3) is generally the region of highest variability among all antibody molecules produced by a given organism. The CDRs are regions of the polypeptide chain arranged linearly in the primary structure, and separated from each other by Framework Regions. The amino terminal (N-terminal) end of the VL chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the VH chain, which includes three CDRs identified as H1, H2 and H3. The majority of amino acid residues in the variable domains, or Fv fragments (VH and VL), are part of the framework regions (approximately 85%). The three dimensional, or tertiary, structure of an antibody molecule is such that the framework regions are more internal to the molecule and provide the majority of the structure, with the CDRs on the external surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, August 1970); Chothia (as described in C. Chothia et al., “Conformations of immunoglobulin hypervariable regions,” Nature, vol. 342, no. 6252, pp. 877-883, December 1989); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55-77, January 2003). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include: 1) Computational predictions of the tertiary structure of the antibody/epitope binding interactions based on the chemical nature of the amino acid sequence of the antibody variable region and composition of the epitope; 2) Hydrogen-deuterium exchange and mass spectroscopy; 3) Polypeptide fragmentation and peptide mapping approaches in which one generates multiple overlapping peptide fragments from the full length of the polypeptide and evaluates the binding affinity of these peptides for the epitope; 4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific VH and VL domains as appropriate.

In certain aspects, affinity matured antibodies are enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992) describes affinity maturation by VH and VL domain shuffling, random mutagenesis of CDR and/or framework residues employed in phage display is described by Rajpal et al., PNAS. 24: 8466-8471 (2005) and Thie et al., Methods Mol Biol. 525:309-22 (2009) in conjugation with computation methods as demonstrated in Tiller et al., Front. Immunol. 8:986 (2017).

Chimeric immunoglobulins are the products of fused genes derived from different species; “humanized” chimeras generally have the framework region (FR) from human immunoglobulins and one or more CDRs are from a non-human source.

In certain aspects, portions of the heavy and/or light chain are identical or homologous to corresponding sequences from another particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity. U.S. Pat. No. 4,816,567; and Morrison et al., Proc. Natl. Acad. Sci. USA 81:6851 (1984). For methods relating to chimeric antibodies, see, e.g., U.S. Pat. No. 4,816,567; and Morrison et al., Proc. Natl. Acad. Sci. USA 81:6851-6855 (1985), each of which are specifically incorporated herein by reference in their entirety. CDR grafting is described, for example, in U.S. Pat. Nos. 6,180,370, 5,693,762, 5,693,761, 5,585,089, and 5,530,101, which are all hereby incorporated by reference for all purposes.

In some embodiments, minimizing the antibody polypeptide sequence from the non-human species optimizes chimeric antibody function and reduces immunogenicity. Specific amino acid residues from non-antigen recognizing regions of the non-human antibody are modified to be homologous to corresponding residues in a human antibody or isotype. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. For use in humans, the V region composed of CDR1, CDR2, and partial CDR3 for both the light and heavy chain variance region from a non-human immunoglobulin, are grafted with a human antibody framework region, replacing the naturally occurring antigen receptors of the human antibody with the non-human CDRs. In some instances, corresponding non-human residues replace framework region residues of the human immunoglobulin. Furthermore, humanized antibodies may comprise residues that are not found in the recipient antibody or in the donor antibody to further refine performance. The humanized antibody may also comprise at least a portion of an immunoglobulin constant region (Fc), typically that of a human immunoglobulin. See, e.g., Jones et al., Nature 321:522 (1986); Riechmann et al., Nature 332:323 (1988); Presta, Curr. Op. Struct. Biol. 2:593 (1992); Vaswani and Hamilton, Ann. Allergy, Asthma and Immunol. 1:105 (1998); Harris, Biochem. Soc. Transactions 23; 1035 (1995); Hurle and Gross, Curr. Op. Biotech. 5:428 (1994); Verhoeyen et al., Science 239:1534-36 (1988).

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

1. Functional Antibody Fragments and Antigen-Binding Fragments

a. Antigen-Binding Fragments

Certain aspects relate to antibody fragments, such as antibody fragments that bind to and/or neutralize inflammatory mediators. The term functional antibody fragment includes antigen-binding fragments of an antibody that retain the ability to specifically bind to an antigen. These fragments are constituted of various arrangements of the variable region heavy chain (VH) and/or light chain (VL); and in some embodiments, include constant region heavy chain 1 (CH1) and light chain (CL). In some embodiments, they lack the Fc region constituted of heavy chain 2 (CH2) and 3 (CH3) domains. Embodiments of antigen binding fragments and the modifications thereof may include: (i) the Fab fragment type constituted with the VL, VH, CL, and CHl domains; (ii) the Fd fragment type constituted with the VH and CHl domains; (iii) the Fv fragment type constituted with the VH and VL domains; (iv) the single domain fragment type, dAb, (Ward, 1989; McCafferty et al., 1990; Holt et al., 2003) constituted with a single VH or VL domain; (v) isolated complementarity determining region (CDR) regions. Such terms are described, for example, in Harlow and Lane, Antibodies: A Laboratory Manual, Cold Spring Harbor Laboratory, NY (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015). The citations in this paragraph are all incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment means a monovalent antigen-binding fragment of an antibody containing the VL, VH, CL and CH1 domains. The term Fab′ fragment means a monovalent antigen-binding fragment of a monoclonal antibody that is larger than a Fab fragment. For example, a Fab′ fragment includes the VL, VH, CL and CH1 domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two VH and VL domains, and can further include all or part of the two CL and CH1 domains.

The term Fd fragment means a fragment of the heavy chain of a monoclonal antibody, which includes all or part of the VH, including the CDRs. An Fd fragment can further include CH1 region sequences.

The term Fv fragment means a monovalent antigen-binding fragment of a monoclonal antibody, including all or part of the VL and VH, and absent of the CL and CH1 domains. The VL and VH include, for example, the CDRs. Single-chain antibodies (sFv or scFv) are Fv molecules in which the VL and VH regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv)2 means bivalent or bispecific sFv polypeptide chains that include oligomerization domains at their C-termini, separated from the sFv by a hinge region (Pack et al. 1992). The oligomerization domain comprises self-associating a-helices, e.g., leucine zippers, which can be further stabilized by additional disulfide bonds. (scFv)2 fragments are also known as “miniantibodies” or “minibodies.”

A single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens.

b. Fragment Crystallizable Region, Fc

An Fc region contains two heavy chain fragments comprising the CH2 and CH3 domains of an antibody. The two heavy chain fragments are held together by two or more disulfide bonds and by hydrophobic interactions of the CH3 domains. The term “Fc polypeptide” as used herein includes native and mutein forms of polypeptides derived from the Fc region of an antibody. Truncated forms of such polypeptides containing the hinge region that promotes dimerization are included.

c. Polypeptides with Antibody CDRs & Scaffolding Domains that Display the CDRs

Antigen-binding peptide scaffolds, such as complementarity-determining regions (CDRs), are used to generate protein-binding molecules in accordance with the embodiments. Generally, a person skilled in the art can determine the type of protein scaffold on which to graft at least one of the CDRs. It is known that scaffolds, optimally, must meet a number of criteria such as: good phylogenetic conservation; known three-dimensional structure; small size; few or no post-transcriptional modifications; and/or be easy to produce, express, and purify. Skerra, J Mol Recognit, 13:167-87 (2000).

The protein scaffolds can be sourced from, but not limited to: fibronectin type III FN3 domain (known as “monobodies”), fibronectin type III domain 10, lipocalin, anticalin, Z-domain of protein A of Staphylococcus aureus, thioredoxin A or proteins with a repeated motif such as the “ankyrin repeat”, the “armadillo repeat”, the “leucine-rich repeat” and the “tetratricopeptide repeat”. Such proteins are described in US Patent Publication Nos. 2010/0285564, 2006/0058510, 2006/0088908, 2005/0106660, and PCT Publication No. WO2006/056464, each of which are specifically incorporated herein by reference in their entirety. Scaffolds derived from toxins from scorpions, insects, plants, mollusks, etc., and the protein inhibiters of neuronal NO synthase (PIN) may also be used.

B. Chimeric Antigen Receptor

In some embodiments, the CD70 targeting agent comprises a CD70-specific CAR molecule or cells, such as T cells comprising and/or expressing a CD70-specific CAR molecule. Chimeric antigen receptor T cells, or CAR T cells, are T cells from either patient, donor, or produced in vitro, that are genetically modified to express chimeric receptors specific to a tumor antigen, along with a signaling domain and co-stimulatory molecules. This fusion of the antibody-derived single chain variable fragment with the T cell intracellular signaling domains endows the CAR T cell with the ability to recognize the tumor antigen in a non-MHC-restricted manner.

A CAR molecule typically comprises one or more antibody binding region(s), an extracellular spacer, a transmembrane domain, and a cytoplasmic region. These are further described below.

1. Antigen Binding Regions

The antigen-binding region may be a single-chain variable fragment (scFv) derived from a CD70 antibody. “Single-chain Fv” or “scFv” antibody fragments comprise the V_{H} and V_{L} domains of an antibody, wherein these domains are present in a single polypeptide chain. In some embodiments, the antigen-binding domain further comprises a peptide linker between the VH and VL domains, which may facilitate the scFv forming the desired structure for antigen binding.

The variable regions of the antigen-binding domains of the polypeptides of the disclosure can be modified by mutating amino acid residues within the VH and/or VL CDR 1, CDR 2 and/or CDR 3 regions to improve one or more binding properties (e.g., affinity) of the antibody. The term “CDR” refers to a complementarity-determining region that is based on a part of the variable chains in immunoglobulins (antibodies) and T-cell receptors, generated by B cells and T cells respectively, where these molecules bind to their specific antigen. Since most sequence variation associated with immunoglobulins and T-cell receptors is found in the CDRs, these regions are sometimes referred to as hypervariable regions. Mutations may be introduced by site-directed mutagenesis or PCR-mediated mutagenesis and the effect on antibody binding, or other functional property of interest, can be evaluated in appropriate in vitro or in vivo assays. Preferably conservative modifications are introduced and typically no more than one, two, three, four or five residues within a CDR region are altered. The mutations may be amino acid substitutions, additions or deletions.

Framework modifications can be made to the antibodies to decrease immunogenicity, for example, by “backmutating” one or more framework residues to the corresponding germline sequence.

It is also contemplated that the antigen binding domain may be multi-specific or multivalent by multimerizing the antigen binding domain with VH and VL region pairs that bind either the same antigen (multi-valent) or a different antigen (multi-specific).

2. Extracellular Spacer

An extracellular spacer may link the antigen-binding domain to the transmembrane domain. It should be flexible enough to allow the antigen-binding domain to orient in different directions to facilitate antigen binding. In one embodiment, the spacer is the hinge region from IgG. Alternatives include the CH2CH3 region of immunoglobulin and portions of CD3.

As used herein, the term “hinge” refers to a flexible polypeptide connector region (also referred to herein as “hinge region” or “spacer”) providing structural flexibility and spacing to flanking polypeptide regions and can consist of natural or synthetic polypeptides. A “hinge” derived from an immunoglobulin (e.g., IgGl) is generally defined as stretching from Glu216 to Pro230 of human IgGl (Burton (1985) Molec. Immunol., 22: 161-206). Hinge regions of other IgG isotypes may be aligned with the IgG1 sequence by placing the first and last cysteine residues forming inter-heavy chain disulfide (S—S) bonds in the same positions. The hinge region may be of natural occurrence or non-natural occurrence, including but not limited to an altered hinge region as described in U.S. Pat. No. 5,677,425. The hinge region can include a complete hinge region derived from an antibody of a different class or subclass from that of the CH1 domain. The term “hinge” can also include regions derived from CD8 and other receptors that provide a similar function in providing flexibility and spacing to flanking regions.

3. Transmembrane Domain

The transmembrane domain is a hydrophobic alpha helix that spans the membrane. Different transmembrane domains may result in different receptor stability.

The transmembrane domain is interposed between the extracellular spacer and the cytoplasmic region. In some embodiments, the transmembrane domain is interposed between the extracellular spacer and one or more costimulatory regions. In some embodiments, a linker is between the transmembrane domain and the one or more costimulatory regions. In some embodiments, the transmembrane domain is derived from CD28, CD8, CD4, CD3-zeta, CD134, or CD7.

4. Cytoplasmic Region

After antigen recognition, receptors cluster and a signal is transmitted to the cell through the cytoplasmic region. In some embodiments, the costimulatory domains described herein are part of the cytoplasmic region.

Cytoplasmic regions and/or costimulatory regions suitable for use in the polypeptides of the disclosure include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation by way of binding of the antigen to the antigen binding domain. In some embodiments, the cytoplasmic region includes at least one (e.g., one, two, three, four, five, six, etc.) ITAM motif as described herein. In some embodiments, the cytoplasmic region includes DAP10/CD28 type signaling chains.

Cytoplasmic regions suitable for use in the polypeptides of the disclosure include immunoreceptor tyrosine-based activation motif (ITAM)-containing intracellular signaling polypeptides. An ITAM motif is YX₁X₂(L/I), where X₁ and X₂ are independently any amino acid. In some cases, the cytoplasmic region comprises 1, 2, 3, 4, or 5 ITAM motifs. In some cases, an ITAM motif is repeated twice in an endodomain, where the first and second instances of the ITAM motif are separated from one another by 6 to 8 amino acids, e.g., (YX₁X₂(L/I))(X3)ₙ₍YX₁X₂(L/I)), where n is an integer from 6 to 8, and each of the 6-8 X₃ can be any amino acid.

A suitable cytoplasmic region may be an FFAM motif-containing portion that is derived from a polypeptide that contains an ITAM motif. For example, a suitable cytoplasmic region can be an ITAM motif-containing domain from any ITAM motif-containing protein. Thus, a suitable endodomain need not contain the entire sequence of the entire protein from which it is derived. Examples of suitable ITAM motif-containing polypeptides include, but are not limited to: DAP12, DAP10, FCER1G (Fc epsilon receptor I gamma chain); CD3D (CD3 delta); CD3E (CD3 epsilon); CD3G (CD3 gamma); CD3-zeta; and CD79A (antigen receptor complex-associated protein alpha chain).

Non-limiting examples of suitable costimulatory regions, such as those included in the cytoplasmic region, include, but are not limited to, polypeptides from 4-1BB (CD137), CD28, ICOS, OX-40, BTLA, CD27, CD30, GITR, and HVEM.

C. Bi-Specific T Cell Engager (BiTE)

Bispecific T cell engagers are a new class of immunotherapeutic molecules intended for the treatment of cancer. These molecules, termed BiTEs, enhance the patient's immune response to tumors by retargeting T cells to tumor cells. BiTEs comprise two single chain variable fragments (scFv) connected in tandem by a flexible linker. This structure and specificity allows a BiTE to physically link a T cell to a tumor cell, ultimately stimulating T cell activation, tumor killing and cytokine production. Embodiments include BiTEs comprising a CD70-specific targeting region, such as a CD70-specific scFV. The BiTE may further comprise specificity to a further cancer-associated molecule, such as EGFR or AXL. Accordingly, embodiments of the disclosure relate to BiTEs comprising a CD70-specific scFV and an EGFR-specific scFv. Further embodiments of the disclosure relate to BiTEs comprising a CD70-specific scFv and an AXL scFV. Further embodiments of the disclosure relate to BiTEs comprising a CD70-specific scFv and a tumor antigen-specific scFv. In some embodiments, the tumor antigen comprises a tumor antigen associated with non small cell lung cancer. Further embodiments relate to a BiTE comprising a CD70-specific targeting region and a TCR-specific targeting region. For example, the TCR-specific targeting region may target a TCR subunit on T cells, such as CD3.

D. Tri-Specific Natural Killer Cell Engager Therapy (TriNKET)

TriNKETs include a NK cell activation region and an antigen binding region, wherein the antigen binding region binds to CD70. TriNKETs are designed to crosslink tumors and NK cells. In some embodiments, the NK cell activation region comprises a NK-activation molecule, such as a cell surface molecule that can activate the cell. The NK cell activation region and/or antigen binding region may comprise a scFv specific for a NK activation protein and an scFv specific for a cancer antigen, respectively. In some embodiments, the TriNKET comprises a scFv domain specific for CD16. In some embodiments, the TriNKET comprises a scFV that specifically binds to CD70. The TriNKET may further comprise an IL-15 or IL-2 molecule. TriNKETs may serve to (a) to direct NK cells to tumors by facilitating formation of intracellular synapses; (b) to bind CD16 on NK cells to trigger ADCC; and (c) to drive in vivo NK cell expansion through IL-15 or IL-2 expression.

### III. CELLS

Certain embodiments relate to cells comprising polypeptides or nucleic acids of the disclosure, such as CD70-targeting agents. In some embodiments the cell is an immune cell or a T cell. “T cell” includes all types of immune cells expressing CD3 including T-helper cells, cytotoxic T-cells, T-regulatory cells (Treg) gamma-delta T cells, natural-killer (NK) cells, and neutrophils. The T cell may refer to a CD4+ or CD8+ T cell.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), human embryonic kidney (HEK) 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), HLHepG2 cells, Hut-78, Jurkat, HL-60, NK cell lines (e.g., NKL, NK92, and YTS), and the like.

In some instances, the cell is not an immortalized cell line, but is instead a cell (e.g., a primary cell) obtained from an individual. For example, in some cases, the cell is an immune cell obtained from an individual. As an example, the cell is a T lymphocyte obtained from an individual. As another example, the cell is a cytotoxic cell obtained from an individual. As another example, the cell is a stem cell or progenitor cell obtained from an individual. In some embodiments, the cell used in therapy of a patient is autologous. In some embodiments, the cell used in therapy of a patient is non-autologous.

### IV. METHODS FOR MODIFYING GENOMIC DNA

In certain embodiments, the genomic DNA is modified either to include additional mutations, insertions, or deletions, or to integrate certain molecular constructs of the disclosure so that the constructs are expressed from the genomic DNA. In some embodiments, a nucleic acid encoding a polypeptide of the disclosure is integrated into the genomic DNA of a cell. In some embodiments, the integration is targeted integration. In some embodiments, targeted integration is achieved through the use of a DNA digesting agent/polynucleotide modification enzyme, such as a site-specific recombinase and/or a targeting endonuclease. The term “DNA digesting agent” refers to an agent that is capable of cleaving bonds (i.e. phosphodiester bonds) between the nucleotide subunits of nucleic acids. One specific target is the TRAC (T-cell receptor alpha constant) locus. For instance, cells would first be electroporated with a ribonucleoprotein (RNP) complex consisting of Cas9 protein complexed with a single-guide RNA (sgRNA) targeting the TRAC (T-cell receptor alpha constant) locus. Fifteen minutes post electroporation, the cells would be treated with AAV6 carrying the HDR template that encodes for the CAR.

Therefore, one aspect, the current disclosure includes targeted integration. One way of achieving this is through the use of an exogenous nucleic acid sequence (i.e., a landing pad) comprising at least one recognition sequence for at least one polynucleotide modification enzyme, such as a site-specific recombinase and/or a targeting endonuclease. Site-specific recombinases are well known in the art, and may be generally referred to as invertases, resolvases, or integrases. Non-limiting examples of site-specific recombinases may include lambda integrase, Cre recombinase, FLP recombinase, gamma-delta resolvase, Tn3 resolvase, ΦC31 integrase, Bxb1-integrase, and R4 integrase. Site-specific recombinases recognize specific recognition sequences (or recognition sites) or variants thereof, all of which are well known in the art. For example, Cre recombinases recognize LoxP sites and FLP recombinases recognize FRT sites.

Contemplated targeting endonucleases include zinc finger nucleases (ZFNs), meganucleases, transcription activator-like effector nucleases (TALENs), CRIPSR/Cas-like endonucleases, I-Tevl nucleases or related monomeric hybrids, or artificial targeted DNA double strand break inducing agents. Exemplary targeting endonucleases is further described below. For example, typically, a zinc finger nuclease comprises a DNA binding domain (i.e., zinc finger) and a cleavage domain (i.e., nuclease), both of which are described below. Also included in the definition of polynucleotide modification enzymes are any other useful fusion proteins known to those of skill in the art, such as may comprise a DNA binding domain and a nuclease.

A landing pad sequence is a nucleotide sequence comprising at least one recognition sequence that is selectively bound and modified by a specific polynucleotide modification enzyme such as a site-specific recombinase and/or a targeting endonuclease. In general, the recognition sequence(s) in the landing pad sequence does not exist endogenously in the genome of the cell to be modified. For example, where the cell to be modified is a CHO cell, the recognition sequence in the landing pad sequence is not present in the endogenous CHO genome. The rate of targeted integration may be improved by selecting a recognition sequence for a high efficiency nucleotide modifying enzyme that does not exist endogenously within the genome of the targeted cell. Selection of a recognition sequence that does not exist endogenously also reduces potential off-target integration. In other aspects, use of a recognition sequence that is native in the cell to be modified may be desirable. For example, where multiple recognition sequences are employed in the landing pad sequence, one or more may be exogenous, and one or more may be native.

One of ordinary skill in the art can readily determine sequences bound and cut by site-specific recombinases and/or targeting endonucleases.

Multiple recognition sequences may be present in a single landing pad, allowing the landing pad to be targeted sequentially by two or more polynucleotide modification enzymes such that two or more unique nucleic acids (comprising, among other things, receptor genes and/or inducible reporters) can be inserted. Alternatively, the presence of multiple recognition sequences in the landing pad, allows multiple copies of the same nucleic acid to be inserted into the landing pad. When two nucleic acids are targeted to a single landing pad, the landing pad includes a first recognition sequence for a first polynucleotide modification enzyme (such as a first ZFN pair), and a second recognition sequence for a second polynucleotide modification enzyme (such as a second ZFN pair). Alternatively, or additionally, individual landing pads comprising one or more recognition sequences may be integrated at multiple locations. Increased protein expression may be observed in cells transformed with multiple copies of a payload Alternatively, multiple gene products may be expressed simultaneously when multiple unique nucleic acid sequences comprising different expression cassettes are inserted, whether in the same or a different landing pad. Regardless of the number and type of nucleic acid, when the targeting endonuclease is a ZFN, exemplary ZFN pairs include hSIRT, hRSK4, and hAAVS1, with accompanying recognition sequences.

Generally speaking, a landing pad used to facilitate targeted integration may comprise at least one recognition sequence. For example, a landing pad may comprise at least one, at least two, at least three, at least four, at least five, at least six, at least seven, at least eight, at least nine, or at least ten or more recognition sequences. In embodiments comprising more than one recognition sequence, the recognition sequences may be unique from one another (i.e. recognized by different polynucleotide modification enzymes), the same repeated sequence, or a combination of repeated and unique sequences.

One of ordinary skill in the art will readily understand that an exogenous nucleic acid used as a landing pad may also include other sequences in addition to the recognition sequence(s). For example, it may be expedient to include one or more sequences encoding selectable or screenable genes as described herein, such as antibiotic resistance genes, metabolic selection markers, or fluorescence proteins. Use of other supplemental sequences such as transcription regulatory and control elements (i.e., promoters, partial promoters, promoter traps, start codons, enhancers, introns, insulators and other expression elements) can also be present.

In addition to selection of an appropriate recognition sequence(s), selection of a targeting endonuclease with a high cutting efficiency also improves the rate of targeted integration of the landing pad(s). Cutting efficiency of targeting endonucleases can be determined using methods well-known in the art including, for example, using assays such as a CEL-1 assay or direct sequencing of insertions/deletions (Indels) in PCR amplicons.

The type of targeting endonuclease used in the methods and cells disclosed herein can and will vary. The targeting endonuclease may be a naturally-occurring protein or an engineered protein. One example of a targeting endonuclease is a zinc-finger nuclease, which is discussed in further detail below.

Another example of a targeting endonuclease that can be used is an RNA-guided endonuclease comprising at least one nuclear localization signal, which permits entry of the endonuclease into the nuclei of eukaryotic cells. The RNA-guided endonuclease also comprises at least one nuclease domain and at least one domain that interacts with a guiding RNA. An RNA-guided endonuclease is directed to a specific chromosomal sequence by a guiding RNA such that the RNA-guided endonuclease cleaves the specific chromosomal sequence. Since the guiding RNA provides the specificity for the targeted cleavage, the endonuclease of the RNA-guided endonuclease is universal and may be used with different guiding RNAs to cleave different target chromosomal sequences. Discussed in further detail below are exemplary RNA-guided endonuclease proteins. For example, the RNA-guided endonuclease can be a CRISPR/Cas protein or a CRISPR/Cas-like fusion protein, an RNA-guided endonuclease derived from a clustered regularly interspersed short palindromic repeats (CRISPR)/CRISPR-associated (Cas) system.

The targeting endonuclease can also be a meganuclease. Meganucleases are endodeoxyribonucleases characterized by a large recognition site, i.e., the recognition site generally ranges from about 12 base pairs to about 40 base pairs. As a consequence of this requirement, the recognition site generally occurs only once in any given genome. Among meganucleases, the family of homing endonucleases named “LAGLIDADG” has become a valuable tool for the study of genomes and genome engineering. Meganucleases may be targeted to specific chromosomal sequence by modifying their recognition sequence using techniques well known to those skilled in the art. See, for example, Epinat et al., 2003, Nuc. Acid Res., 31(11):2952-62 and Stoddard, 2005, Quarterly Review of Biophysics, pp. 1-47.

Yet another example of a targeting endonuclease that can be used is a transcription activator-like effector (TALE) nuclease. TALEs are transcription factors from the plant pathogen Xanthomonas that may be readily engineered to bind new DNA targets. TALEs or truncated versions thereof may be linked to the catalytic domain of endonucleases such as FokI to create targeting endonuclease called TALE nucleases or TALENs. See, e.g., Sanjana et al., 2012, Nature Protocols 7(1):171-192; Bogdanove A J, Voytas D F., 2011, Science, 333(6051):1843-6; Bradley P, Bogdanove A J, Stoddard B L., 2013, Curr Opin Struct Biol., 23(1):93-9.

Another exemplary targeting endonuclease is a site-specific nuclease. In particular, the site-specific nuclease may be a “rare-cutter” endonuclease whose recognition sequence occurs rarely in a genome. Preferably, the recognition sequence of the site-specific nuclease occurs only once in a genome. Alternatively, the targeting nuclease may be an artificial targeted DNA double strand break inducing agent.

In some embodiments, targeted integrated can be achieved through the use of an integrase. For example, The phiC31 integrase is a sequence-specific recombinase encoded within the genome of the bacteriophage phiC31. The phiC31 integrase mediates recombination between two 34 base pair sequences termed attachment sites (att), one found in the phage and the other in the bacterial host. This serine integrase has been show to function efficiently in many different cell types including mammalian cells. In the presence of phiC31 integrase, an attB-containing donor plasmid can be unidirectional integrated into a target genome through recombination at sites with sequence similarity to the native attP site (termed pseudo-attP sites). phiC31 integrase can integrate a plasmid of any size, as a single copy, and requires no cofactors. The integrated transgenes are stably expressed and heritable.

In one embodiment, genomic integration of polynucleotides of the disclosure is achieved through the use of a transposase. For example, a synthetic DNA transposon (e.g. “Sleeping Beauty” transposon system) designed to introduce precisely defined DNA sequences into the chromosome of vertebrate animals can be used. The Sleeping Beauty transposon system is composed of a Sleeping Beauty (SB) transposase and a transposon that was designed to insert specific sequences of DNA into genomes of vertebrate animals. DNA transposons translocate from one DNA site to another in a simple, cut-and-paste manner. Transposition is a precise process in which a defined DNA segment is excised from one DNA molecule and moved to another site in the same or different DNA molecule or genome.

As do all other Tc1/mariner-type transposases, SB transposase inserts a transposon into a TA dinucleotide base pair in a recipient DNA sequence. The insertion site can be elsewhere in the same DNA molecule, or in another DNA molecule (or chromosome). In mammalian genomes, including humans, there are approximately 200 million TA sites. The TA insertion site is duplicated in the process of transposon integration. This duplication of the TA sequence is a hallmark of transposition and used to ascertain the mechanism in some experiments. The transposase can be encoded either within the transposon or the transposase can be supplied by another source, in which case the transposon becomes a non-autonomous element. Non-autonomous transposons are most useful as genetic tools because after insertion they cannot independently continue to excise and re-insert. All of the DNA transposons identified in the human genome and other mammalian genomes are non-autonomous because even though they contain transposase genes, the genes are non-functional and unable to generate a transposase that can mobilize the transposon.

### I. Methods of Treatment

Aspects of the current disclosure relate to methods for treating cancer, such as non-small cell lung cancer. In further embodiments, the CD70 targeting molecules described herein may be used for stimulating an immune response. The immune response stimulation may be done in vitro, in vivo, or ex vivo. In some embodiments, the the CD70 targeting molecules described herein are for preventing relapse. The method generally involves administering a CD70 targeting molecule to a patient. In some embodiments, the CD70 targeting molecule is a genetically modified mammalian cell with an expression vector, or an RNA (e.g., in vitro transcribed RNA), comprising nucleotide sequences encoding a polypeptide that target CD70. The cell can be an immune cell (e.g., a T lymphocyte or NK cell), a stem cell, a progenitor cell, etc. In some embodiments, the cell is a cell described herein or the progeny thereof.

Embodiments of the disclosure include ex vivo methods. For example, a T lymphocyte, a stem cell, or an NK cell (or cell described herein) is obtained from an individual; and the cell obtained from the individual is genetically modified to express a CD70 targeting molecule of the disclosure. In some cases, the genetically modified cell is activated ex vivo. In other cases, the genetically modified cell is introduced into an individual (e.g., the individual from whom the cell was obtained); and the genetically modified cell is activated in vivo.

In some embodiments, the methods relate to administration of the cells or CD70 targeting molecules for the treatment of a cancer or administration to a person with a cancer. In some embodiments, the cancer is non-small cell lung cancer.

### II. Pharmaceutical Compositions

The present disclosure includes methods for treating disease and modulating immune responses in a subject in need thereof. The disclosure includes cells that may be in the form of a pharmaceutical composition that can be used to induce or modify an immune response.

Administration of the compositions according to the current disclosure will typically be via any common route. This includes, but is not limited to parenteral, orthotopic, intradermal, subcutaneous, intramuscular, intraperitoneal, or intravenous injection.

Typically, compositions of the disclosure are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of pharmaceutical compositions comprising cellular components are applicable. The dosage of the pharmaceutical composition will depend on the route of administration and will vary according to the size and health of the subject.

In many instances, it will be desirable to have multiple administrations of at most about or at least about 3, 4, 5, 6, 7, 8, 9, 10 or more. The administrations may range from 2-day to 12-week intervals, more usually from one to two week intervals. The course of the administrations may be followed by assays for alloreactive immune responses and T cell activity.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. The pharmaceutical compositions of the current disclosure are pharmaceutically acceptable compositions.

The compositions of the disclosure can be formulated for parenteral administration, e.g., formulated for injection via the intravenous, intramuscular, sub-cutaneous, or even intraperitoneal routes. Typically, such compositions can be prepared as injectables, either as liquid solutions or suspensions and the preparations can also be emulsified.

The pharmaceutical forms suitable for injectable use include sterile aqueous solutions or dispersions; formulations including sesame oil, peanut oil, or aqueous propylene glycol. It also should be stable under the conditions of manufacture and storage and must be preserved against the contaminating action of microorganisms, such as bacteria and fungi.

Sterile injectable solutions are prepared by incorporating the active ingredients (i.e. cells of the disclosure) in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

An effective amount of a composition is determined based on the intended goal. The term “unit dose” or “dosage” refers to physically discrete units suitable for use in a subject, each unit containing a predetermined quantity of the composition calculated to produce the desired responses discussed herein in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

### V. ADDITIONAL THERAPIES

The current methods and compositions of the disclosure may include one or more additional therapies known in the art and/or described herein. In some embodiments, the additional therapy or agent comprises an additional cancer treatment. Examples of such treatments are described herein.

A. Immunotherapies

In some embodiments, the additional therapy or agent comprises a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies are known in the art, and some are described below.

1. Inhibition of Co-Stimulatory Molecules

In some embodiments, the immunotherapy comprises an inhibitor of a co-stimulatory molecule. In some embodiments, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

2. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

3. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some embodiments, the CAR-T therapy targets CD19.

4. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

5. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death.

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

6. Checkpoint Inhibitors and Combination Treatment

In some embodiments, the additional therapy or agent comprises immune checkpoint inhibitors. Certain embodiments are further described below.

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO0 1/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

B. Oncolytic Virus

In some embodiments, the additional therapy or agent comprises an oncolytic virus. An oncolytic virus is a virus that preferentially infects and kills cancer cells. As the infected cancer cells are destroyed by oncolysis, they release new infectious virus particles or virions to help destroy the remaining tumour. Oncolytic viruses are thought not only to cause direct destruction of the tumour cells, but also to stimulate host anti-tumour immune responses for long-term immunotherapy

C. Polysaccharides

In some embodiments, the additional therapy or agent comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

D. Neoantigens

In some embodiments, the additional therapy or agent comprises neoantigen administration. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors.

E. Chemotherapies

In some embodiments, the additional therapy or agent or agent comprises a chemotherapy. Suitable classes of chemotherapeutic agents include (a) Alkylating Agents, such as nitrogen mustards (e.g., mechlorethamine, cyclophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydrazine derivatives (e.g., procarbazine), and adrenocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain embodiments. In some embodiments, the amount of cisplatin delivered to the cell and/or subject in conjunction with the construct comprising an Egr-1 promoter operably linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluorouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

F. Radiotherapy

In some embodiments, the additional therapy or agent or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some embodiments, the amount of ionizing radiation is greater than 20 Gy and is administered in one dose. In some embodiments, the amount of ionizing radiation is 18 Gy and is administered in three doses. In some embodiments, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some embodiments, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some embodiments, the amount of IR may be presented as a total dose of IR, which is then administered in fractionated doses. For example, in some embodiments, the total dose is 50 Gy administered in 10 fractionated doses of 5 Gy each. In some embodiments, the total dose is 50-90 Gy, administered in 20-60 fractionated doses of 2-3 Gy each. In some embodiments, the total dose of IR is at least, at most, or about 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some embodiments, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some embodiments, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some embodiments, at least, at most, or exactly 1, 2, 3, 4, 5, 6, 7, 8, 9, 10, 11, or 12 (or any derivable range therein) fractionated doses are administered per day. In some embodiments, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

G. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

H. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### VI. ADMINISTRATION OF THERAPEUTIC COMPOSITIONS

Methods of the disclosure include administration of a combination of therapeutic agents and/or administration of therapeutic agents, such as fecal matter and therapeutic regimens, such as steroid therapy or anti-integrin therapy, for example. The therapy may be administered in any suitable manner known in the art. For example, the therapies may be administered sequentially (at different times) or concurrently (at the same time). In some embodiments, the therapies are in a separate composition. In some embodiments, the therapies are in the same composition.

Various combinations of the therapies may be employed, for example, one therapy designated “A” and another therapy designated “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

The therapies of the disclosure, such as the fecal matter from a healthy subject may be administered by the same route of administration or by different routes of administration. In some embodiments, the therapy is administered intracolonically, intravenously, intramuscularly, subcutaneously, topically, orally, transdermally, intraperitoneally, intraorbitally, by implantation, by inhalation, intrathecally, intraventricularly, or intranasally. In some embodiments, the microbial modulator is administered intravenously, intramuscularly, subcutaneously, topically, orally, transdermally, intraperitoneally, intraorbitally, by implantation, by inhalation, intrathecally, intraventricularly, or intranasally.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In some embodiments, the therapeutically effective or sufficient amount of a therapeutic composition that is administered to a human will be in the range of about 0.01 to about 50 mg/kg of patient body weight whether by one or more administrations. In some embodiments, the therapeutic agent used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In some embodiments, the therapeutic agent is administered at 15 mg/kg. However, other dosage regimens may be useful. In one embodiment, a therapeutic agent described herein is administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### VII. KITS

Certain aspects of the disclosure also encompass kits for performing the methods of the disclosure, such as detection of, diagnosis of, or treatment of cancer. Such kits can be prepared from readily available materials and reagents. For example, such kits can comprise any one or more of the following materials: enzymes, reaction tubes, buffers, detergent, primers, probes, antibodies. In a preferred embodiment, these kits allow a practitioner to obtain samples of neoplastic cells in blood, tears, semen, saliva, urine, tissue, serum, stool, sputum, cerebrospinal fluid and supernatant from cell lysate. In another preferred embodiment these kits include the needed apparatus for performing RNA extraction, RT-PCR, and gel electrophoresis. Instructions for performing the assays can also be included in the kits.

The kits may further comprise instructions for using the kit for assessing sequences, means for converting and/or analyzing sequence data to generate prognosis. The agents in the kit for measuring biomarker expression may comprise a plurality of PCR probes and/or primers for qRT-PCR and/or a plurality of antibody or fragments thereof for assessing expression of the biomarkers. In another embodiment, the agents in the kit for measuring biomarker expression may comprise an array of polynucleotides complementary to the mRNAs of the biomarkers of the invention. Possible means for converting the expression data into expression values and for analyzing the expression values to generate scores that predict survival or prognosis may be also included.

Kits may comprise a container with a label. Suitable containers include, for example, bottles, vials, and test tubes. The containers may be formed from a variety of materials such as glass or plastic. The container may hold a composition which includes a probe that is useful for prognostic or non-prognostic applications, such as described above. The label on the container may indicate that the composition is used for a specific prognostic or non-prognostic application, and may also indicate directions for either in vivo or in vitro use, such as those described above. The kit may comprise the container described above and one or more other containers comprising materials desirable from a commercial and user standpoint, including buffers, diluents, filters, needles, syringes, and package inserts with instructions for use.

Further kit embodiments relate to kits comprising the therapeutic compositions of the disclosure. The kits may be useful in the treatment methods of the disclosure and comprise instructions for use.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### EGFR TKI Resistance is Associated with a Mesenchymal Phenotype and Increased Expression of CD70

Here, the inventors demonstrate that CD70 is a therapeutic target for EGFR mutant, TKI-resistant tumors, and approaches targeting CD70 such as CD70-antibody drug conjugates, anti-CD70 CAR-T cell, or TriNKETs, EGFR-CD70 BiTEs, Axl-CD70 BiTEs, or other approaches targeting CD70 (collectively referred to as CD70-directed therapies) alone or in combination with other treatments may be effective for EGFR mutant, TKI resistant tumors. Furthermore, EGFR mutations are a biomarker for the selection of patients to be treated with agents targeting CD70. In addition, the inventors describe that targeting CD70 is a therapeutic strategy for mesenchymal NSCLC tumors and that a mesenchymal status as determined by gene expression or protein markers (collectively referred to as epithelial to mesenchymal transition (EMT) biomarkers) is a biomarker for selecting patients for treatment with CD70-targeting therapies.

In effort to identify potential targets in EGFR mutant, TKI resistant NSCLC, the inventors derived a panel of NSCLC cell lines with acquired resistance to EGFR TKIs, erlotinib, gefitinib, and osimertinib. Transcriptomic and proteomic profiling revealed that resistant cells had undergone an epithelial to mesenchymal transition (EMT). Gene expression analysis revealed that CD70 was significantly overexpressed in EGFR TKI resistant cells compared to parental (EGFR TKI sensitive) cells. The inventors' finding that gene expression of CD70 was highly upregulated in NSCLC cells with acquired resistant to EGFR TKIs was validated by flow cytometry, demonstrating that resistant cells express higher levels of CD70 protein on the cell surface compared to parental (EGFR TKI sensitive) cells. To evaluate whether CD70 is increased in NSCLC clinical specimens that have undergone EMT, the inventors evaluated RNAseq data from the TCGA. CD70 expression correlated with a mesenchymal gene signature in NSCLC tumor specimens.

CD70 is known to be expressed on T and B cells and also on some malignant cells including leukemia cells and renal cell carcinomas. It is thought that CD70 expression contributes to an immunosuppressive environment by affecting/attracting regulatory T cells, promoting T cell apoptosis and exhaustion. In addition, tumor cells expressing CD70 can be directly targeted using anti-CD70 antibody-drug conjugates or CAR T-cells. Collectively, the data presented herein demonstrates that CD70 is overexpressed in NSCLC cells with acquired resistance to EGFR TKIs and suggest that CD70 targeting may be an effective therapeutic strategy in this setting.

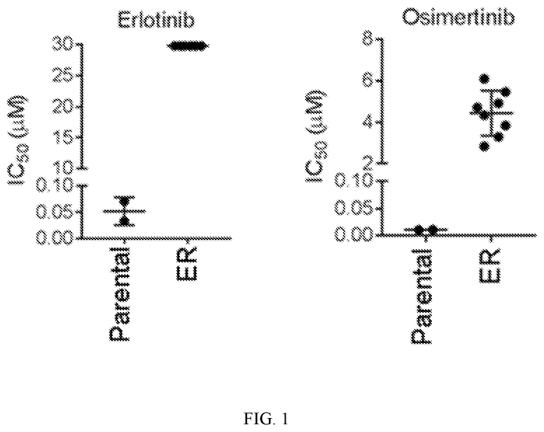

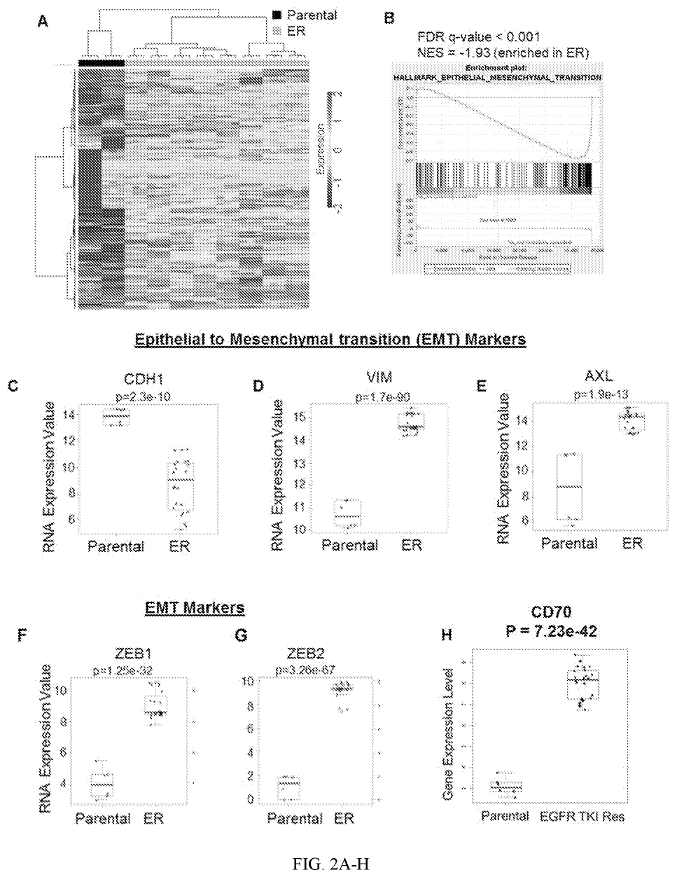

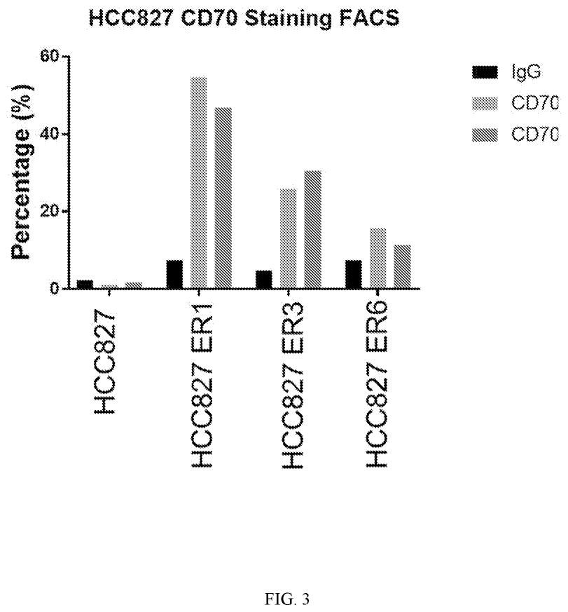

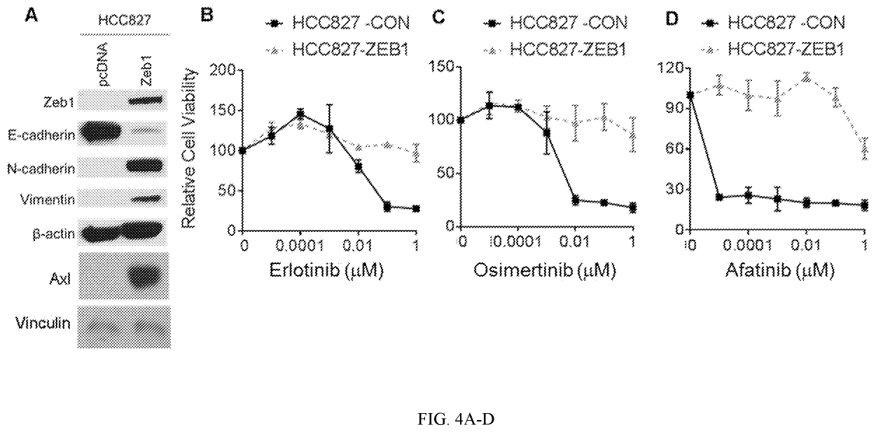

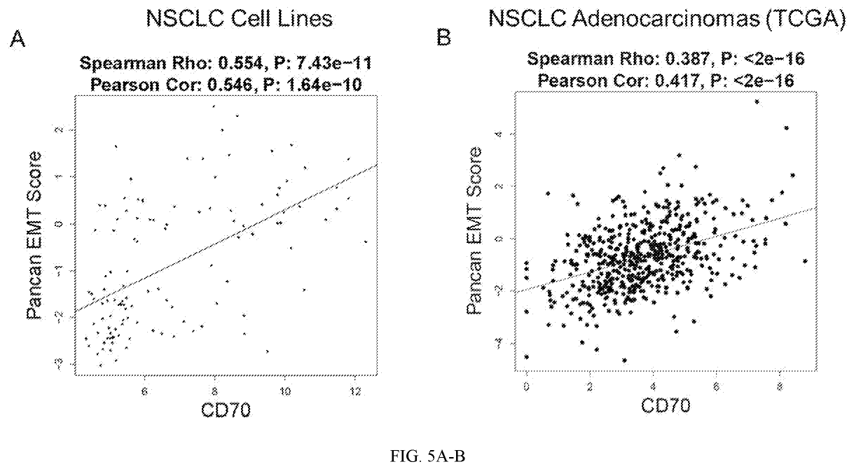

While EGFR mutant NSCLC patients are initially responsive to EGFR tyrosine kinase inhibitors (TKI), resistant disease inevitably emerges. The inventors derived a panel of NSCLC cell lines with acquired resistance to EGFR TKIs. EGFR-TKI resistant (ER) cells were negative for secondary EGFR mutations and were resistant to EGFR TKIs (FIG. 1). Using RNAseq and gene expression analysis, the inventors determined that EGFR TKI resistant cells exhibited a mesenchymal gene expression signature including loss of CDH1 expression and increased expression of VIM and AXL as well as ZEB1 and ZEB2, key mediators of epithelial to mesenchymal transition (EMT) (FIG. 2A-G). The RNA expression analysis further revealed that EGFR TKI resistant cells highly overexpressed CD70 (FIG. 2H). Flow cytometry analysis revealed increased protein levels of CD70 on the surface of EGFR TKI resistant cells compared to EGFR TKI sensitive parental cells (FIG. 3). Moreover, induction of EMT through forced expression of ZEB1 in HCC827 parental (EGFR TKI sensitive) cells was sufficient to rendered cells resistant to EGFR inhibition with erlotinib, osimertinib or afatinib (FIG. 4). Given the findings that EGFR TKI resistance is associated with EMT and that these cells overexpress CD70, the inventors next evaluated whether CD70 expression was associated with a mesenchymal phenotype in human lung adenocarcinomas using the TCGA database. The inventors found that CD70 expression was significantly associated with an EMT gene expression signature in lung adenocarcinomas and in an extensive panel of NSCLC cell lines (FIG. 5). CD70 is typically expressed on T-cells and B-cells, but can also be expressed by some malignant cells. Expression of CD70 by tumor cells is thought to contribute to an immunosuppressive environment by affecting/attracting regulatory T cells and promoting T cell apoptosis and exhaustion. These findings that CD70 expression is enhanced in EGFR TKI resistant cells suggests that targeting CD70 can be clinically useful in the setting of EGFR TKI resistant NSCLC.

### Example 2

### EGFR TKI Resistance is Associated with a Mesenchymal Phenotype and Increased Expression of CD70

This example may contain duplicated and/or reorganized data from Example 1.

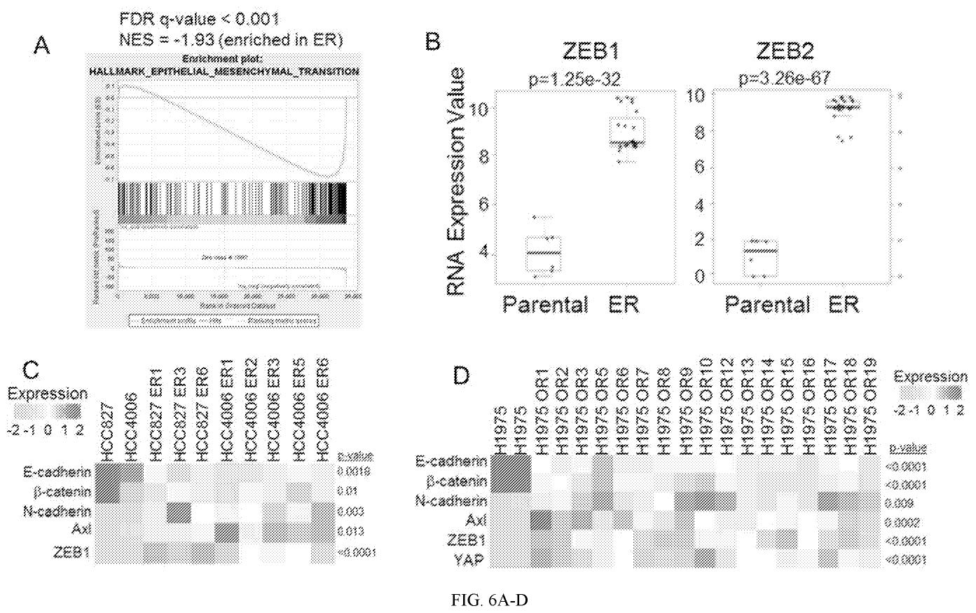

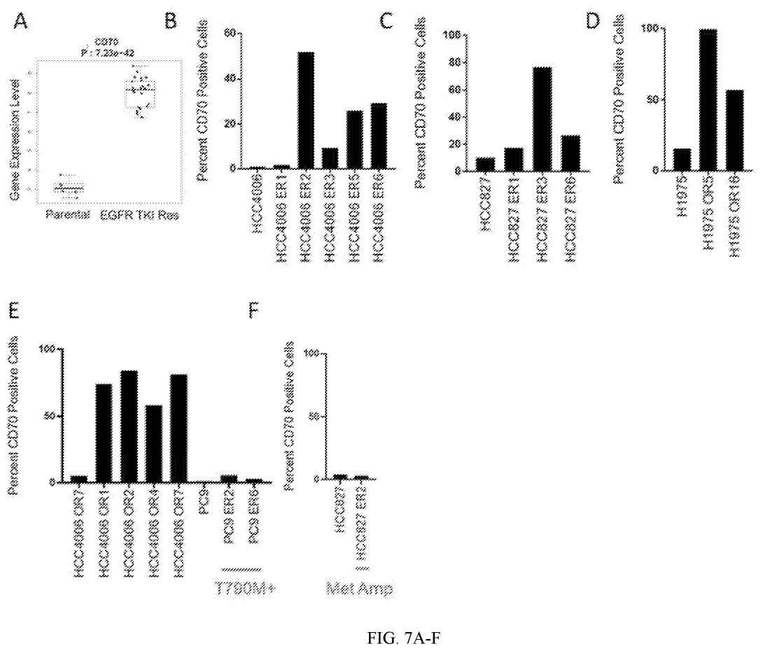

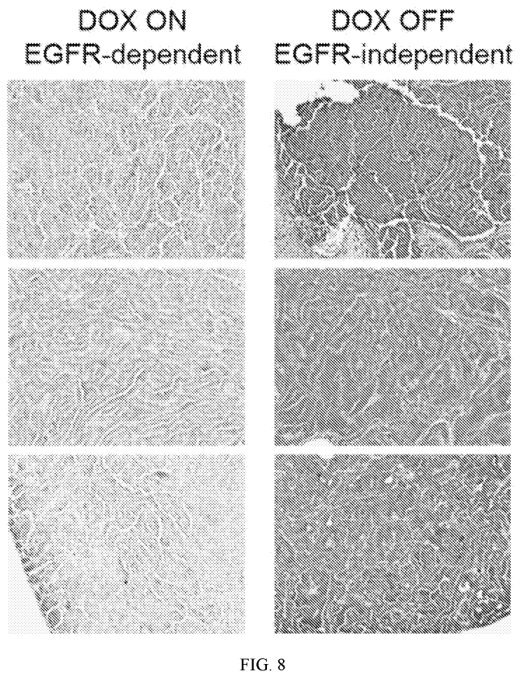

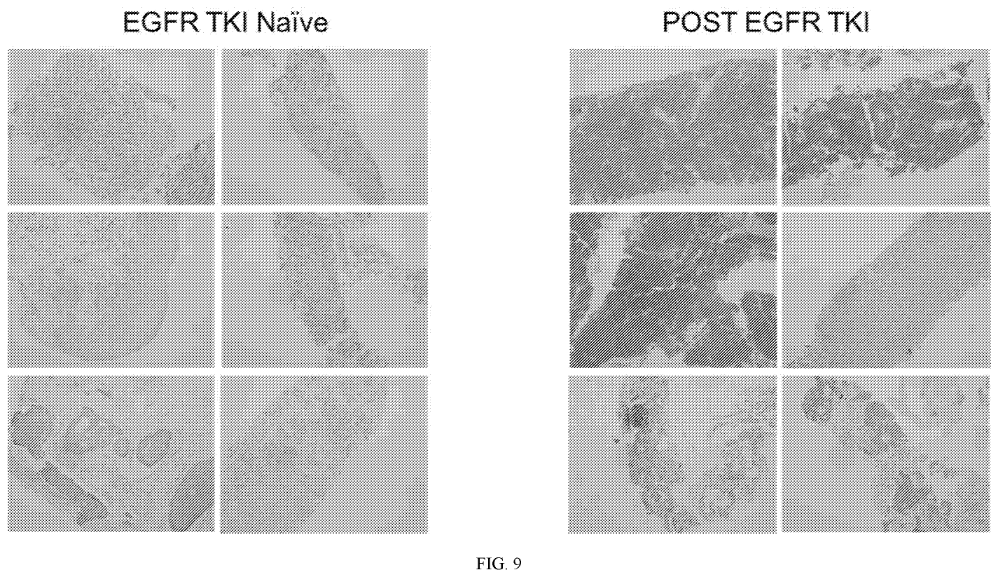

While EGFR mutant NSCLC patients are initially responsive to EGFR tyrosine kinase inhibitors (TKI), resistant disease inevitably emerges. The inventors derived a panel of NSCLC cell lines with acquired resistance to EGFR TKIs. EGFR-TKI resistant (ER) cells were negative for secondary EGFR mutations and were resistant to EGFR TKIs (FIG. 1). Using RNAseq and gene expression analysis, the inventors determined that EGFR TKI resistant cells exhibited a mesenchymal gene expression signature including loss of CDH1 expression and increased expression of VIM and AXL as well as ZEB1 and ZEB2, key mediators of epithelial to mesenchymal transition (EMT) (FIG. 6). The RNA expression analysis further revealed that EGFR TKI resistant cells highly overexpressed CD70 (FIG. 7A). Flow cytometry analysis revealed increased protein levels of CD70 on the surface of EGFR TKI resistant cells compared to EGFR TKI sensitive parental cells (FIG. 7B-F). To determine whether CD70 is elevated in animal models of EGFR TKI resistance, the inventors utilized a doxycycline-inducible EGFR L858R GEMM model in which administration of doxycycline results in mutant EGFR expression and the development of lung tumors. Once tumors were visualized by CT imaging, doxycycline was withdrawn from a subset of animals to mimic EGFR inhibition. After a period of tumor regression, tumors began to regrow as determine by CT imaging. Animals were treated with osimertinib to confirm an EGFR TKI resistant phenotype. Tumors were collected an CD70 expression was analyzed by immunohistochemistry. CD70 expression was elevated in tumors with acquired EGFR-independence (FIG. 8). Next, the inventors evaluated CD70 expression in EGFR mutant EGFRTKI naïve NSCLC clinical specimens and EGFR mutant NSCLC specimens collected after EGFR TKI resistance. While CD70 expression was minimal in treatment naïve tissues, CD70 was highly expressed in EGFR TKI resistant tumors (FIG. 9).

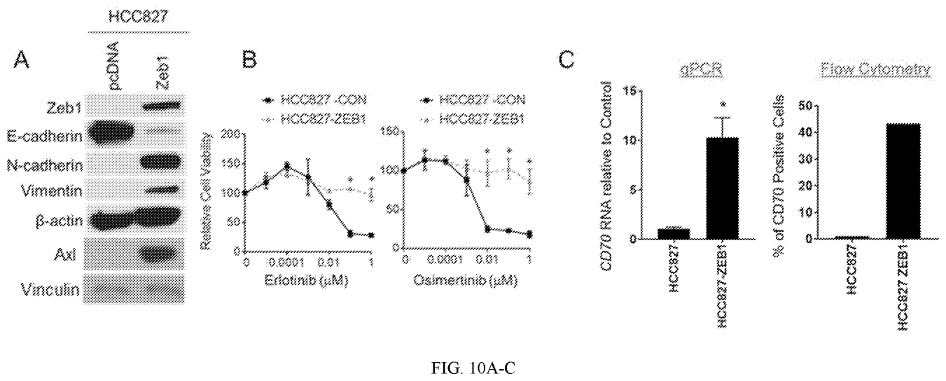

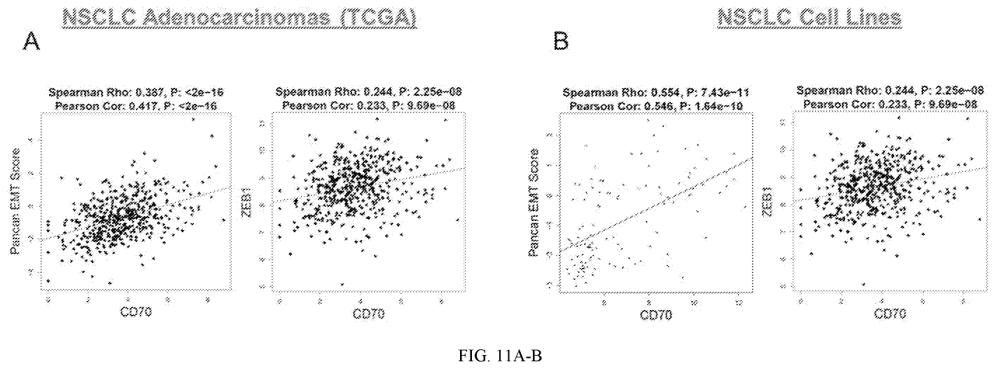

Next, the inventors investigated the impact of EMT on CD70 expression in EGFR mutant NSCLC cell lines. The inventors induced EMT through forced expression of ZEB1 in HCC827 parental (EGFR TKI sensitive) cells. ZEB1 expression induced a mesenchymal phenotype and was sufficient to rendered cells resistant to EGFR inhibition with erlotinib, osimertinib or afatinib (FIGS. 10A&B). ZEB1 expression induced a significant increase in CD70 mRNA levels and cell surface expression of CD70. The inventors next evaluated whether CD70 expression was associated with a mesenchymal phenotype in NSCLC cell lines and human lung adenocarcinomas using the TCGA database. The inventors found that CD70 expression was significantly associated with an EMT gene expression signature and ZEB1 expression in lung adenocarcinomas and in an extensive panel of NSCLC cell lines (FIG. 11).

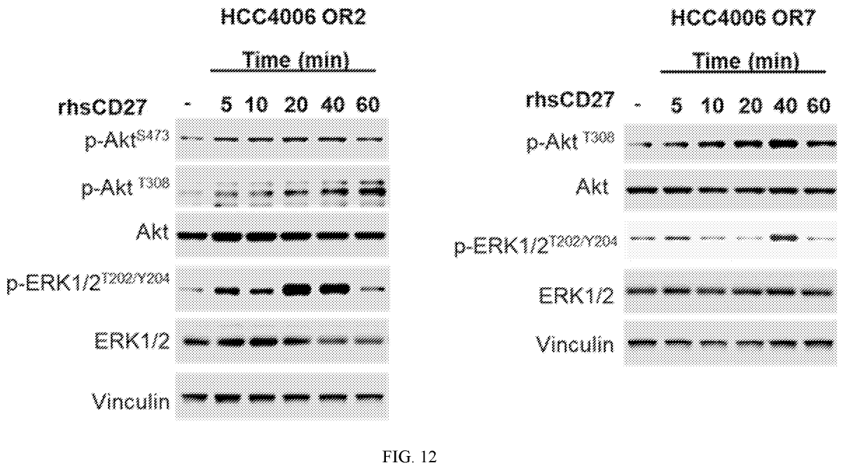

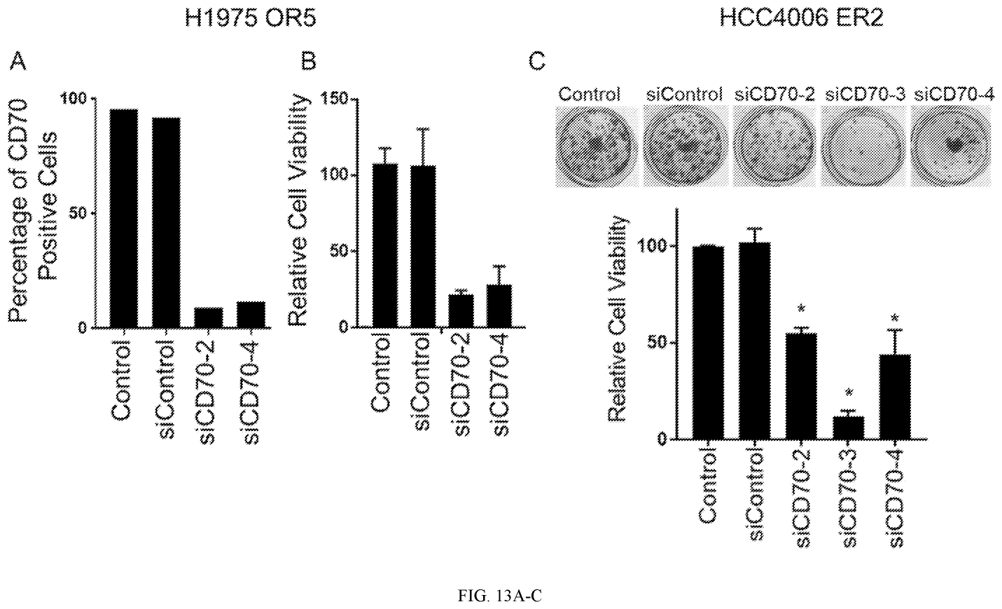

Binding of CD27 to CD70 induces activation of signal transduction pathways downstream of CD70. To investigate the potential impact of CD70 signaling on EGFR TKI resistant cells, the inventors stimulated EGR TKI resistant cells with recombinant soluble CD27. CD27 treatment resulted in activation of Akt and ERK, important signal transduction molecules known to be re-activated in EGFR TKI resistance (FIG. 12). Next, the inventors used siRNA to knockdown CD70 expression and found that knockdown of CD70 impaired the growth of EGFR TKI resistant cells by clonogenic assay (FIG. 13).

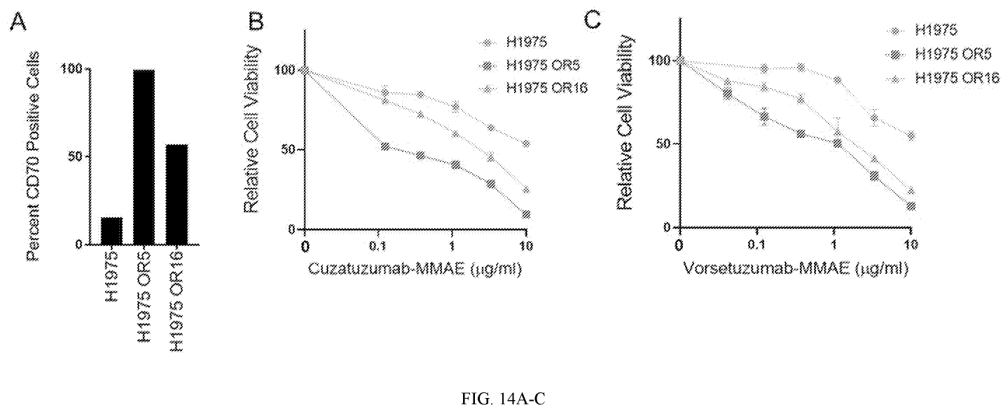

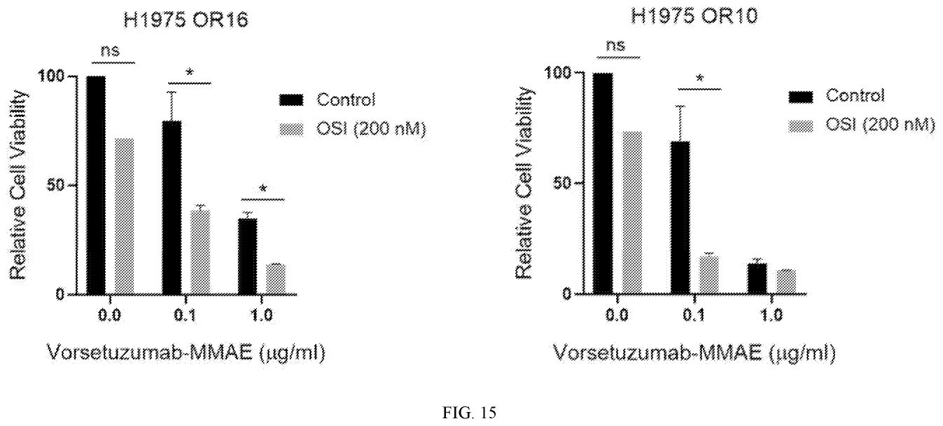

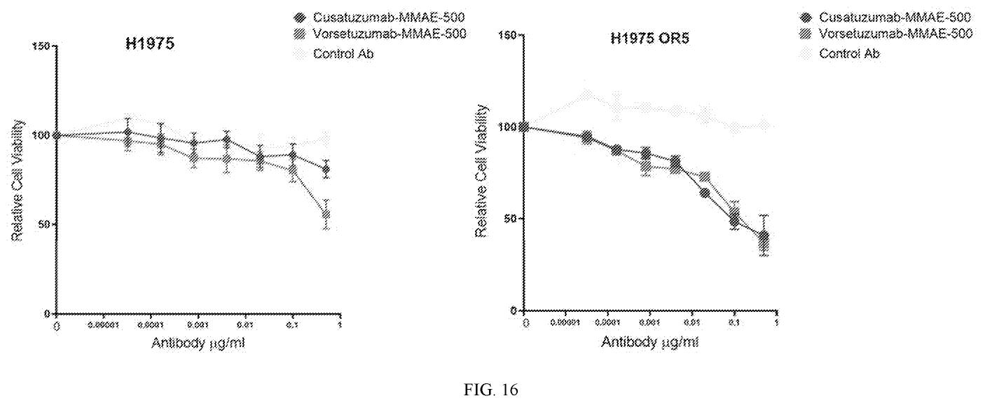

To determine whether CD70 antibody drug conjugates (ADCs) are a valid approach for targeting EGFR TKI resistant cells, the inventors treated H1975 cells (CD70 low and EGFR TKI sensitive) and H1975 OR5 and H1975 OR16 (both EGFR TKI resistant and CD70 high) with increasing concentrations of the CD70 ADCs cuzatuzumab-MMAE or vorsetuzumab-MMAE. As expected, H1975 OR5 and OR16 cells were more sensitive to CD70 ADCs than H1975 parental cells (FIG. 14 and FIG. 16). The inventors further observed an additive anti-tumor cell effect when osimertinib was combined with anti-CD70 ADCs. (FIG. 15).

CD70 is typically expressed on T-cells and B-cells but can also be expressed by some malignant cells. Expression of CD70 by tumor cells is thought to contribute to an immunosuppressive environment by affecting/attracting regulatory T cells and promoting T cell apoptosis and exhaustion. These findings that CD70 expression is enhanced in EGFR TKI resistant cells suggests that targeting CD70 may be clinically useful in the setting of EGFR TKI resistant NSCLC.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. The publications listed in the application, to the extent that they provide exemplary procedural or other details supplementary to those set forth herein, are specifically incorporated herein by reference.

What is claimed is:

1. A method for treating EGFR-mutant non-small-cell lung cancer (NSCLC) in a patient comprising administering a CD70 targeting molecule to the patient.

2. A method for treating an epithelial-to-mesenchymal transition (EMT)-positive NSCLC in a patient comprising administering a CD70-targeting molecule to the patient.

3. The method of claim 1 or 2, wherein the patient has been determined to have EGFR mutant NSCLC.

4. The method of any one of claims 1-3, wherein the NSCLC comprises lung adenocarcinoma.

5. The method of any one of claims 1-4, wherein the patient is a non-smoker.

6. The method of any one of claims 1-5, wherein the EGFR mutant comprises an activating mutation.

7. The method of claim 6, wherein the activating mutation comprises L858R or a deletion in exon 19.

8. The method of any one of claims 1-7, wherein the EGFR mutation comprises a Class I, II, or III EGFR mutation.

9. The method of any one of claims 1-8, wherein the patient has not been tested for CD70 expression in cancer cells.

10. The method of any one of claims 1-8, wherein the patient has been determined to have CD70-expressing cancer cells.

11. The method of any one of claims 1-10, wherein the patient has been previously treated for NSCLC.

12. The method of claim 11, wherein the patient has been determined to have acquired resistance to the previous treatment.

13. The method of claim 11 or 12, wherein the previous treatment comprises EGFR tyrosine kinase inhibitor (TKI) therapy and wherein the therapy comprises one or more EGFR TKIs.

14. The method of any one of claims 11-13, wherein the previous treatment comprises single-agent EGFR TKI therapy.

15. The method of any one of claims 11-13, wherein the previous treatment comprises a combination of at least two EGFR TKIs.

16. The method of any one of claims 11-15, wherein the patient was determined to have systemic disease progression while receiving continuous EGFR TKI therapy.

17. The method of any one of claims 13-16, wherein the EGFR TKI therapy comprises one or more of gefitinib, erlotinib, afatinib, dacomitinib, osimertinib, and brigatinib.

18. The method of any one of claims 1-17, wherein the method further comprises administration of an additional therapy.

19. The method of claim 18, wherein the additional therapy comprises chemotherapy, radiation, surgery, TKI therapy, or an immunotherapy.

20. The method of claim 18 or 19, wherein the additional therapy comprises one or more of durvalumab, atezolizumab, pembrolizumab, nivolumab, necitumumab, and bevacizumab.

21. The method of any one of claims 18-20, wherein the additional therapy comprises one or more of carboplatin, pemetrexed, nab-paclitaxel, photofrin, cisplatin, docetaxel, gemcitabine, paclitaxel, and vinorelbine.

22. The method of any one of claims 18-21, wherein the additional therapy comprises one or more of alectinib, lorlatinib, and ceritinib.

23. The method of any one of claims 18-22, wherein the additional therapy comprises one or more of gefitinib, erlotinib, afatinib, dacomitinib, osimertinib, and brigatinib.

24. The method of claim 23, wherein the additional therapy comprises osimertinib.

25. The method of any one of claims 1-22, wherein the method further comprises administration of adjuvant and/or neo-adjuvant therapy.

26. The method of any one of claims 1-25, wherein the patient has been determined to be ALK mutant.

27. The method of any one of claims 1-25, wherein the patient has been determined to not be ALK mutant.

28. The method of any one of claims 1-27, wherein the CD70 targeting molecule comprises an anti-CD70 antibody or a CD70-binding fragment thereof.

29. The method of claim 28, wherein the additional therapy comprises a secondary antibody linked to a toxic molecule.

30. The method of claim 29, wherein the secondary antibody and toxic molecule are linked through a cleavable linker.

31. The method of any one of claims 28-30, wherein the antibody is humanized or chimeric.

32. The method of claim any one of claims 28-31, wherein the antibody comprises cusatuzumab or vorsetuzumab.

33. The method of any one of claims 28-32, wherein the antibody is conjugated to a molecule.

34. The method of claim 33, wherein the molecule is a toxic molecule.

35. The method of claim 34, wherein the toxic molecule comprises monomethyl auristatin E (MMAE), monomethyl auristatin F (MMAF), Pyrrolobenzodiazepine (PBD), or duocarmycin.

36. The method of claim 34 or 35, wherein the CD70 targeting molecule comprises cusatuzumab-MMAE, vorsetuzumab-MMAE, or combinations thereof.

37. The method of any one of claims 1-36, wherein the CD70 targeting molecule comprises a heavy chain variable region and/or a light chain variable region from a CD70 antibody.

38. The method of any one of claims 1-37, wherein the CD70 targeting molecule comprises a CDR1, CDR2, and CDR3 from a heavy chain variable region and/or a CDR1, CDR2, and CDR3 from a light chain variable region.

39. The method of any one of claims 1-38, wherein the CD70 targeting molecule comprises a single chain variable fragment (scFV).

40. The method of any one of claims 1-39, wherein the CD70 targeting molecule comprises a bi-specific T cell engager (BiTE), a chimeric antigen receptor (CAR), a T cell comprising a CAR, or a tri-specific natural killer cell engager therapy (TriNKET).

41. The method of claim 40, wherein the CD70 targeting molecule comprises a cell comprising a BiTE, CAR, or TriNKET.

42. The method of claim 41, wherein the cell comprises a stem cell, a progenitor cell, an immune cell, or a natural killer (NK) cell.

43. The cell of claim 42, wherein the cell comprises a hematopoietic stem or progenitor cell, a T cell, a cell differentiated from mesenchymal stem cells (MSCs) or an induced pluripotent stem cell (iPSC).

44. The cell of claim 42 or 43, wherein the cell is isolated or derived from peripheral blood mononuclear cell (PBMCs).

45. The cell of claim 43 or 44, wherein the T cell comprises a cytotoxic T lymphocyte (CTL), a CD8⁺ T cell, a CD4⁺ T cell, an invariant NK T (iNKT) cell, a gamma-delta T cell, a NKT cell, or a regulatory T cell.

46. The method of any one of claims 40-45, wherein the CD70 targeting molecule comprises CTX130 or ALLO-316.

47. The method of any one of claims 40-45, wherein the CD70 targeting molecule comprises a CD27 CAR.

48. The method of any one of claims 1-39, wherein the CD70 targeting molecule comprises SGN-75, SGN-CD70A, AMG 172, and/or ARGX-110.

49. The method of any one of claims 1-48, wherein a biological sample from the patient has been determined to be positive for one or more EMT markers.

50. The method of claim 49, wherein the biological sample comprises tumor cells and/or tumor-associated cells.

51. The method of claim 49 or 50, wherein the biological sample comprises a biopsy.

52. The method of any one of claims 49-51, wherein the one or more EMT markers comprise a reduction of an epithelial marker and/or an increase of a mesenchymal marker.

53. The method of claim 49 or 52, wherein the EMT markers comprise one or more of CDH1, VIM, AXL, ZEB1, and ZEB2.

54. A composition comprising a CD70 targeting molecule and one or more additional therapeutic agent(s).

55. The composition of claim 54, wherein the additional therapeutic agent comprises chemotherapy, radiation, surgery, TKI therapy, an immunotherapy, or combinations thereof.

56. The composition of claim 54 or 55, wherein the additional therapeutic agent comprises one or more of durvalumab, atezolizumab, pembrolizumab, nivolumab, necitumumab, and bevacizumab.

57. The composition of any one of claims 54-56, wherein the additional therapeutic agent comprises one or more of carboplatin, pemetrexed, nab-paclitaxel, photofrin, cisplatin, docetaxel, gemcitabine, paclitaxel, and vinorelbine.

58. The composition of any one of claims 55-57, wherein the additional therapeutic agent comprises one or more of alectinib, lorlatinib, and ceritinib.

59. The composition of any one of claims 55-58, wherein the additional therapeutic agent comprises one or more of gefitinib, erlotinib, afatinib, dacomitinib, osimertinib, and brigatinib.

60. The composition of claim 59, wherein the additional therapeutic agent comprises osimertinib.

61. The composition of any one of claims 55-57, wherein the CD70 targeting molecule comprises an anti-CD70 antibody or a CD70-binding fragment thereof.

62. The composition of claim 61, wherein the additional therapeutic agent comprises a secondary antibody linked to a toxic molecule.

63. The composition of claim 62, wherein the secondary antibody and toxic molecule are linked through a cleavable linker.

64. The composition of any one of claims 61-63, wherein the antibody is humanized or chimeric.

65. The composition of claim any one of claims 61-64, wherein the antibody comprises cusatuzumab or vorsetuzumab.

66. The composition of any one of claims 61-65, wherein the antibody is conjugated to a molecule.

67. The composition of claim 66, wherein the molecule is a toxic molecule.

68. The composition of claim 67, wherein the toxic molecule comprises monomethyl auristatin E (MMAE), duocarmycin, monomethyl auristatin F (MMAF), or pyrrolobenzodiazepine (PBD).

69. The composition of claim 67 or 68, wherein the CD70 targeting molecule comprises cusatuzumab-MMAE, vorsetuzumab-MMAE, or combinations thereof.

70. The composition of any one of claims 54-69, wherein the CD70 targeting molecule comprises a heavy chain variable region and/or a light chain variable region from a CD70 antibody.

71. The composition of any one of claims 54-70, wherein the CD70 targeting molecule comprises a CDR1, CDR2, and CDR3 from a heavy chain variable region and/or a CDR1, CDR2, and CDR3 from a light chain variable region.

72. The composition of any one of claims 54-71, wherein the CD70 targeting molecule comprises a single chain variable fragment (scFV) that specifically binds to CD70.

73. The composition of any one of claims 54-72, wherein the CD70 targeting molecule comprises a bi-specific T cell engager (BiTE), a chimeric antigen receptor (CAR), a T cell comprising a CAR, or a tri-specific natural killer cell engager therapy (TriNKET).

74. The composition of any one of claims 54-73, wherein the CD70 targeting molecule comprises SGN-75, SGN-CD70A, AMG 172, and/or ARGX-110.
